(19) United States
(12) Patent Application Publication
(10) Pub. No.: US 2022/0339297 A1
(43) Pub. Date: Oct. 27, 2022

(54) HUMAN GENE THERAPY METHODS FOR HEMOPHILIA A

(71) Applicant: Bayer HealthCare LLC, Whippany, NJ (US)

(72) Inventors: Nicole Schmidt, Berlin (DE); Inge Ivens, Nevada City, CA (US); Anita Shah, Whippany, NJ (US); Konstantina Vanevski, Basel (CH)

(73) Assignee: Bayer HealthCare LLC, Whippany, NJ (US)

(21) Appl. No.: 17/725,086

(22) Filed: Apr. 20, 2022

Related U.S. Application Data
(60) Provisional application No. 63/178,955, filed on Apr. 23, 2021.
(60) Provisional application No. 63/178,962, filed on Apr. 23, 2021.
(60) Provisional application No. 63/220,281, filed on Jul. 9, 2021.
(60) Provisional application No. 63/220,307, filed on Jul. 9, 2021.
(60) Provisional application No. 63/236,068, filed on Aug. 23, 2021.

(51) Int. Cl. A61K 48/00; A61K 38/37; A61K 9/00; A61P 7/04; C12N 15/861

(52) U.S. Cl. CPC A61K 48/0058; A61K 38/37; A61K 9/0019; A61K 48/0075; A61P 7/04; C12N 15/861; C12N 2750/14132

(57) ABSTRACT

Methods and materials for effective dosages of AAV gene therapy for the treatment and prophylaxis of hemophilia A.

### FIELD OF THE INVENTION

The present invention concerns human gene therapy for treatment and prophylaxis of hemophilia A and related blood coagulation disorders.

### SEQUENCE LISTING SUBMISSION

The Sequence Listing associated with this application is filed in electronic format via EFS-Web and hereby incorporated by reference into the specification in its entirety. The name of the text file containing the Sequence Listing is “139921_00188 Sequence Listing ST25”. The size of the text file is 101,121 bytes, and the text file was created on Apr. 18, 2022.

### BACKGROUND

Hemophilia A (HA or HemA) is the most common inherited bleeding disorder.

According to the US Centers for Disease Control and Prevention, hemophilia A occurs in approximately 1 in 5,000 live male births. Centers for Disease Control and Prevention website, www.cdc.gov/ncbddd/hemophilia/, accessed Apr. 7, 2021. There are about 20,000 people with hemophilia A in the US. Hemophilia A is four times as common as hemophilia B, and more than half of patients with hemophilia A have the severe form of hemophilia A. HA is caused by a deficiency of factor VIII (FVIII) and is well suited for a gene replacement approach, primarily because a modest increase in the level of FVIII (>1% of normal) can ameliorate the severe bleeding phenotype. Adeno-associated viral (AAV) vectors currently show great promise for gene therapy applications because of their excellent safety profile and ability to direct long-term transgene expression from post-mitotic tissues such as the liver. Further analysis of gene therapy to treat HA is found in Machin N et al., “Gene therapy in hemophilia A: a cost-effectiveness analysis,” Blood Adv 2018; 2:1792-1798.

The use of AAV vectors for HA gene therapy poses challenges because of the distinct molecular and biochemical properties of human FVIII (“hFVIII”). Compared with other proteins of similar size, expression of hFVIII is highly inefficient. Bioengineering of the FVIII molecule has resulted in improvement of the FVIII expression. For instance, the hFVIII B domain, which is not required for co-factor activity, has been deleted and replaced by a short 14 amino acid linker that is SFSQNPPVLKRHQR (SEQ ID NO: 21) to create a FVIII variant known as FVIII BDD SQ or BDD SQ, resulting in a 17-fold increase in mRNA levels over full-length wild-type FVIII and a 30% increase in secreted protein. See Ward, Natalie J., et al. “Codon optimization of human factor VIII cDNAs leads to high-level expression.” Blood 117.3 (2011): 798-807 and U.S. Pat. No. 9,393,323 (Nathwani et al.). Recombinant FVIII BDD SQ is in clinical use as a replacement recombinant FVIII product (REFACTO recombinant antihemophilic factor, Wyeth Pharma; XYNTHA recombinant antihemophilic factor, Pfizer).

Another obstacle to AAV-mediated gene transfer for HA gene therapy is the size of the FVIII coding sequence, which at 7.0 kb far exceeds the normal packaging capacity of AAV vectors.

U.S. Pat. No. 10,888,628 to patentee The Trustees of the University of Pennsylvania entitled “Gene Therapy for Treating Hemophilia A” provides useful AAV vectors for gene therapy for HA and related compositions and methods for treating hemophilia A. U.S. Pat. Application Pub. No. 20200237930 A1 to applicant Spark Therapeutics discloses useful AAV vectors for gene therapy for HA and dosages for administration in humans.

Direct comparison of dosages for different hemophilia A gene therapy approaches is hampered by a number of factors, including the different mouse models of hemophilia A utilized in the experimental studies (immune-competent or -deficient mice). Preclinical evaluations of rAAV8-HLP-codop-hFVIII-V3 in immunocompetent F8^{-/-} mice reported ˜15% of normal hFVIII activity at a dose of 2×10¹¹ vg/kg. McIntosh J et al., “Therapeutic levels of FVIII following a single peripheral vein administration of rAAV vector encoding a novel human factor VIII variant,” Blood 2013; 121:3335-3344. Evaluation of BMN 270 (AAV5-co-BDD-F8) at a dose of 6×10¹² vg/kg demonstrated 4.9% of normal hFVIII activity in Rag2^{-/-} mice and detectable expression in two out of 10 DKO mice (double knockout with mutations in both FVIII and Rag2). Bunting S et al., “Gene Therapy with BMN 270 Results in Therapeutic Levels of FVIII in Mice and Primates and Normalization of Bleeding in Hemophilic Mice,” Mol Ther 2018; 26:496-509. At a higher dose of BMN 270 (2×10¹³ vg/kg) 23.5% of normal hFVIII activity was achieved in DKO mice. In preclinical evaluations of SB-525 (AAV6-co-BDD-F8), levels >330% of normal were seen at a dose of 7.2×10¹² vg/kg in mice that were tolerized to hFVIII (mouse FVIII KO R593C mice contain a hF8-R593C transgene under control of a mouse albumin promoter). Riley B E et al., “Development of an Optimized rAAV2/6 Human Factor 8 cDNA Vector Cassette for Hemophilia a Gene Therapy,” Blood 2016; 128:1173.

The need exists for safe and clinically efficacious methods of administering AAV gene therapy vectors to treat HA, such as safe and effective dosage regimens, such as regimens that provide efficacious levels for prophylaxis of bleeding as shown by suitable pharmacokinetic measures, and methods and products for determining safe and efficacious dosages.

### SUMMARY

Certain embodiments herein concern methods for treating hemophilia A comprising administering to a patient in need thereof a therapeutically effective dose of an AAV gene therapy vector for delivering human FVIII or a variant thereof; wherein sustained human FVIII pro-coagulant activity is achieved as measured 10 months after administration. In one embodiment, the dose is from 0.5×10¹³ to 4×10¹³ genome copies/kg. In another embodiment the dose is selected from the group consisting of 0.5×10¹³, 0.6×10¹³, 0.7×10¹³, 0.8×10¹³, 0.9×10¹³, 1.0×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2.0×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3.0×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, and 4.0×10¹³ genome copies/kg.

Certain embodiments herein concern methods for treating hemophilia A comprising administering to a patient in need thereof a dose, preferably a minimally effective dose, of an AAV gene therapy vector that delivers a human FVIII gene to a patient in need thereof, wherein the dose, preferably the minimally effective dose, is 3×10¹¹ genome copies/kg, and optionally wherein the does, preferably the minimally effective dose, when measured 56 days after dosage provides at least about 20% of normal human FVIII activity. In another embodiment, the invention concerns methods for administering a therapeutically effective dose of an AAV gene therapy vector that delivers a human FVIII gene for treatment of hemophilia A comprising obtaining measurements of FVIII activity in a study of at least 100 male mice having a disease pathology for bleeding and who have received injections of the AAV gene therapy vector and obtaining the minimally effective dose calculated from those measurements; and administering to a patient in need thereof the minimally effective dose of the AAV gene therapy vector, optionally wherein the minimally effective dose when measured 56 days after dosage provides at least about 20% of normal human FVIII activity.

In another embodiment, the invention concerns methods for determining a minimally effective dosage of an AAV gene therapy vector for delivering human FVIII; the method comprising obtaining at least 50 male knock out FVIII mice; injecting the male knock out FVIII mice with an IV tail vein injection of either (a) the AAV gene therapy vector at one of four doses, which doses preferably are 3×10¹¹, 1×10, 3×10¹², or 1×10¹³ GC/kg or (b) a vehicle control; wherein the mice receiving the AAV gene therapy vector are divided into four cohorts and each cohort receives a different one of the four doses; performing a first and a second necropsy; wherein the first necropsy occurs on a first group of mice on a day between 23-33 and the second necropsy occurs on a second group of mice on a day between 51-61 after injection; measuring hFVIII activity with each necropsy; determining peak and long term hFVIII activity from the hFVIII activity measurements; and calculating minimally effective dosage from the peak and long term hFVIII activity.

### BRIEF DESCRIPTION OF THE DRAWINGS

FIG. 1: Plasma hFVIII activity levels and anti-hFVIII IgG titers in pilot dose-ranging study. Male FVIII KO mice (n=10/group) were injected IV with 1.5×10¹³ GC/kg, 5.0×10¹² GC/kg, 1.5×10¹² GC/kg, 5 0.0×10¹¹ GC/kg, 1.5×10¹¹ GC/kg, or 1.5×10¹⁰ GC/kg of AAVhu37.E03.TTR.hFVIIIco-SQ.PA75 or vehicle control (100 μl of PBS). (A) hFVIII activity levels were measured in plasma samples taken throughout the in-life phase of the study and at the time of necropsy by a COATEST assay. Dashed line indicates 5% of normal activity; dotted line indicates 10% of normal activity. (B) Anti-hFVIII IgG titers were measured in plasma samples taken throughout the in-life phase of the study and at the time of necropsy by an anti-hFVIII IgG ELISA. Values that were five-fold over background levels (naïve mouse samples) were considered positive. Negative values are denoted as a titer of 1/50 to enable them to be visualized. Graphs show plots of individual mice, with data points and error bars representing mean±standard error of the mean (SEM) values.

FIG. 2: Plasma hFVIII activity levels in vector administered FVIII KO mice. Male FVIII KO mice (n=10/group) were injected IV with 1×10¹³ GC/kg, 3×10¹² GC/kg, 1×10¹² GC/kg, or 3×10¹¹ GC/kg of AAVhu37.E03.TTR.hFVIIIco-SQ.PA75 or vehicle control. hFVIII activity levels were measured in plasma samples taken throughout the in-life phase of the study and at the time of necropsy by a COATEST assay. Mice were necropsied on day 56 (A) or day 28 (B). Graphs show plots of individual mice, with data points and error bars representing mean±SEM values. Dashed lines indicate 20% of normal activity.

FIG. 3: Plasma anti-hFVIII IgG titers in vector administered FVIII KO mice. Male FVIII KO mice (n=10/group) were injected IV with 1×10¹³ GC/kg, 3×10¹² GC/kg, 1×10¹² GC/kg, or 3×10¹¹ GC/kg of AAVhu37.E03.TTR.hFVIIIco-SQ.PA75 or vehicle control. Anti-hFVIII IgG titers were at the time of necropsy by an anti-hFVIII IgG ELISA. Mice were necropsied on day 56 (A) or day 28 (B). Values that were five-fold over background levels (naïve mouse samples) were considered positive. Negative values are denoted as a titer of 1/50 to enable them to be visualized. Graphs show plots of individual mice.

FIG. 4: ALT, AST, and total bilirubin levels in vector administered FVIII KO mice. Male FVIII KO mice (n=10/group) were injected IV with 1×10¹³ GC/kg, 3×10¹² GC/kg, 1×10¹² GC/kg, or 3×10¹¹ GC/kg of AAVhu37.E03.TTR.hFVIIIco-SQ.PA75 or vehicle control. ALT (A, B), AST (C, D), and total bilirubin (E, F) levels were measured in serum samples taken at the time of necropsy by Antech GLP. Mice were necropsied on day 28 (A, C, and E) or day 56 (B, D, and F). Values are expressed as mean±SEM. Groups administered with vector or vehicle control were compared using a Wilcoxon rank-sum test, *p<0.05.

FIG. 5: Vector genome copies and hFVIII RNA transcript levels in livers from vector administered FVIII KO mice. Male FVIII KO mice (n=10/group) were injected IV with 1×10¹³ GC/kg, 3×10¹² GC/kg, 1×10¹² GC/kg, or 3×10¹¹ GC/kg of AAVhu37.E03.TTR.hFVIIIco-SQ.PA75 or vehicle control. At necropsy, livers were harvested and snap frozen. DNA was extracted for quantification of vector GC. Mice were necropsied on day 28 (A) or day 56 (B). Vector GC values are presented per diploid genome. RNA was extracted for quantification of vector transcript levels. Mice were necropsied on day 28 (C) or day 56 (D). hFVIII RNA copies are presented per 100 ng of RNA. Values are plotted as mean±SEM. ns, not significant, **p<0.01 compared to vehicle; ## p<0.01, ### p<0.001 compared to next lowest dose.

FIGS. 6A and 6B: Table 1. Summary of histopathology findings for vector administered FVIII KO mice. Male FVIII KO mice were injected IV with 1×10¹³ GC/kg, 3×10¹² GC/kg, 1×10¹² GC/kg, or 3×10¹¹ GC/kg of AAVhu37.E03.TTR.hFVIIIco-SQ.PA75 or vehicle control (“N/A” in Table 1). To obtain the results identified in Table 1 for the liver, at necropsy, livers were harvested, fixed using 10% neutral buffered formalin, paraffin embedded, sectioned, and stained for histopathology using H&E stain. An experienced board-certified veterinary pathologist evaluated the liver sections in a blinded manner using pre-determined scoring criteria.

FIG. 7: Total protein levels in vector administered FVIII KO mice. Male FVIII KO mice (n=10/group) were injected IV with 1×10¹³ GC/kg, 3×10¹² GC/kg, 1×10¹² GC/kg, or 3×10¹¹ GC/kg of AAVhu37.E03.TTR.hFVIIIco-SQ.PA75 or vehicle control (100 μl PBS). Total protein levels were measured in serum samples taken at the time of necropsy by the diagnostics company Antech GLP. Mice were necropsied on day 28 (A) or day 56 (B). Values are expressed as mean±SEM. Groups administered with vector or vehicle control were compared using a Wilcoxon rank-sum test, *p<0.05.

FIG. 8: Study design for BAY 2599023 phase ½ dose-finding study (NCT03588299). Eligible patients were enrolled sequentially into four dose cohorts to receive a single intravenous infusion of BAY 2599023, with a minimum of two patients per dose level. Patients are to be followed-up for a total of 5 years to evaluate the safety of BAY 2599023 and its effect on clinical outcome measures.

FIG. 9: Safety Outcomes from BAY 2599023 phase ½ dose-finding study determined from first six patients.

FIG. 10: FVIII levels (chromogenic (BDD plasma)) by patient over time in Cohorts 1, 2 and 3, for first 8 patients.

### DETAILED DESCRIPTION

In one aspect, the present invention relates to methods for the treatment or prophylaxis of hemophilia A and other disorders involving hemostasis where expression of FVIII is desirable by administering gene therapy using an AAV vector in a manner and under suitable conditions to result in clinically effective transduction of the gene therapy vector in the patient.

### Definitions

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides include naturally occurring, synthetic, and intentionally modified or altered polynucleotides (e.g., variant nucleic acid). Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of the protein (FVIII) encoded thereby, and some codons of the nucleic acid variant will be changed which in turn changes the amino acids of the protein (FVIII) encoded thereby.

The term “variant Factor VIII (FVIII)” refers to a modified FVIII which has been genetically altered as compared to unmodified wild-type FVIII or FVIII-BDD. Such a variant can be referred to as a “nucleic acid variant encoding Factor VIII (FVIII).” The term “variant” need not appear in each instance of a reference made to nucleic acid encoding FVIII.

A “variant Factor VIII (FVIII)” can also mean a modified FVIII protein such that the modified protein has an amino acid alteration compared to wild-type FVIII. When comparing activity and/or stability, if the encoded variant FVIII protein retains the B-domain, it is appropriate to compare it to wild-type FVIII; and if the encoded variant FVIII protein has a B-domain deletion, it may be compared to FVIII that also has a B-domain deletion, specifically the variant known as hFVIII BDD SQ or simply as BDD (SEQ ID NO: 3). A variant FVIII can include a portion of the B-domain. For example, hFVIII BDD SQ includes a portion of the B-domain.

A variant FVIII can include an “SQ” sequence set forth as SFSQNPPVLKRHQR (SEQ ID NO: 21). A variant FVIII, such as FVIII-BDD can have all or just a portion of the amino acid sequence SFSQNPPVLKRHQR (SEQ ID NO: 21). For example, a variant FVIII-BDD can have 1, 2, 3, 4, 5, 6, 7, 8, 9, 10, 11, 12 or 13 amino acid residues of SFSQNPPVLKRHQR (SEQ ID NO: 21) included. In some embodiments the variant FVIII includes variants comprising an amino acid sequence of SFSQNPPVLKRHQR (SEQ ID NO: 21) with 1, 2, 3, 4, 5, 6, 7, 8, 9, 10, 11, 12 or 13 internal deletions, and/or an amino acid sequence comprising an amino acid sequence having 1, 2, 3, 4, 5, 6, 7, 8, 9, 10, 11, 12 or 13 amino- or carboxy terminal deletions when compared to a wild type or a variant FVIII amino acid sequence known to encode a protein having FVIII coagulant activity.

The “polypeptides,” “proteins” and “peptides” encoded by the “nucleic acid” or “polynucleotide” sequences,” include full-length native (FVIII) sequences, as with naturally occurring wild-type proteins, as well as functional subsequences, modified forms or sequence variants so long as the subsequence, modified form or variant retain some degree of functionality of the native full-length protein. FVIII functionality is determined by the aPPT assay or Coatest chromogenic assay described herein. In methods and uses of the invention, such polypeptides, proteins and peptides encoded by the nucleic acid sequences can be but are not required to be identical to the endogenous protein that is defective, or whose expression is insufficient, or deficient in the treated mammal.

Non-limiting examples of modifications include one or more nucleotide or amino acid substitutions (e.g., 1-3, 3-5, 5-10, 10-15, 15-20, 20-25, 25-30, 30-40, 40-50, 50-100, 100-150, 150-200, 200-250, 250-500, 500-750, 750-850 or more nucleotides or residues). Preferably, such modifications are from a nucleotide or amino acid sequence known to encode a protein having human FVIII activity.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), selectable marker (e.g., antibiotic resistance), and/or polyadenylation signal.

A viral vector is derived from or based upon one or more nucleic acid elements that comprise a viral genome, such as adeno-associated virus (AAV) vectors.

The term “recombinant,” as a modifier of vector, such as recombinant viral (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “AAV vector” is derived from the wild type genome of a virus, such as AAV by using molecular methods to remove the wild type genome from the virus (e.g., AAV), and replacing with a non-native nucleic acid, such as a codon-optimized nucleic acid encoding FVIII or hFVIII-BDD. Typically, for AAV one or both inverted terminal repeat (ITR) sequences of AAV genome are retained in the AAV vector. A “recombinant” viral vector (e.g., AAV) is distinguished from a viral (e.g., AAV) genome, since all or a part of the viral genome has been replaced with a non-native sequence with respect to the viral (e.g., AAV) genomic nucleic acid such as a codon-optimized nucleic acid encoding FVIII or hFVIII-BDD. Incorporation of a non-native sequence therefore defines the viral vector (e.g., AAV) as a “recombinant” vector, which in the case of AAV can be referred to as a “rAAV vector.”

A recombinant vector (e.g., AAV) sequence can be packaged—referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV.” Such particles include proteins that encapsidate or package the vector genome. Particular examples include viral envelope proteins, and in the case of AAV, capsid proteins.

A vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

A “transgene” is used herein to conveniently refer to a nucleic acid that is intended or has been introduced into a cell or organism. Transgenes include any nucleic acid, such as a gene that encodes a polypeptide or protein (e.g., a codon-optimized nucleic acid encoding FVIII or hFVIII-BDD).

In a cell having a transgene, the transgene has been introduced/transferred by way of vector, such as AAV, “transduction” or “transfection” of the cell. The terms “transduce” and “transfect” refer to introduction of a molecule such as a nucleic acid into a cell or host organism. The transgene may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced protein expressed, or nucleic acid transcribed. For gene therapy uses and methods, a transduced cell can be in a subject.

An “expression control element” refers to nucleic acid sequence(s) that influence expression of an operably linked nucleic acid. Control elements, including expression control elements as set forth herein such as promoters and enhancers, Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans. Expression control can be at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence, e.g., nucleic acid encoding FVIII or hFVIII-BDD. A promoter typically increases an amount expressed from operably linked nucleic acid as compared to an amount expressed when no promoter exists.

An “enhancer” as used herein can refer to a sequence that is located adjacent to the heterologous polynucleotide. Enhancer elements are typically located upstream of a promoter element but also function and can be located downstream of or within a sequence (e.g., a nucleic acid encoding FVIII or hFVIII-BDD). Hence, an enhancer element can be located 100 base pairs, 200 base pairs, or 300 or more base pairs upstream or downstream of a nucleic acid encoding FVIII. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the expression constructs of the invention provides for at least partial tissue tropism for the expression of a nucleic acid encoding FVIII or hFVIII-BDD. Examples of promoters that are active in liver are the TTR promoter; thyroxin binding (TBG) promotor (also referred to as P3 promoter); a shortened version of thyroxin binding globulin (TBG-S1); human alpha 1-antitrypsin (hAAT) promoter; albumin promoter (Miyatake et al. J. Virol., 71:5124-32 (1997)); hepatitis B virus core promoter (Sandig et al., Gene Ther. 3:1002-9 (1996)); alpha-fetoprotein (AFP) promoter (Arbuthnot et al., Hum. Gene. Ther., 7:1503-14 (1996)), among others. An example of an enhancer active in liver is apolipoprotein E (apoE) HCR-1 and HCR-2 (Allan et al., J. Biol. Chem., 272:29113-19 (1997)).

The term “operably linked” means that the regulatory sequences necessary for expression of a coding sequence are placed in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

With reference to nucleic acids of the invention, the term “isolated” refers to a nucleic acid molecule that is separated from one or more sequences with which it is immediately contiguous (in the 5′ and 3′ directions) in the naturally occurring genome (genomic DNA) of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule. Alternatively, this term may refer to a protein which has been sufficiently separated from other proteins with which it would naturally be associated, so as to exist in “substantially pure” form.

The term “isolated” does not exclude combinations produced by the hand of man, for example, a recombinant vector (e.g., rAAV) sequence, or virus particle that packages or encapsidates a vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g. chromatographic methods, agarose or polyacrylamide gel electrophoresis, HPLC).

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple polynucleotide or protein (amino acid) sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous nucleic acids or amino acids, e.g., 6, 7, 8, 9, 10, 11, 12, 13, 14, 15, 16, 17, 18, 19, 20, etc. contiguous nucleic acids or amino acids. In additional embodiments, the length of the sequence sharing identity is 21 or more contiguous nucleic acids or amino acids, e.g., 21, 22, 23, 24, 25, 26, 27, 28, 29, 30, 31, 32, 33, 34, 35, 36, 37, 38, 39, 40, etc. contiguous nucleic acids or amino acids. In further embodiments, the length of the sequence sharing identity is 41 or more contiguous nucleic acids or amino acids, e.g. 42, 43, 44, 45, 45, 47, 48, 49, 50, etc., contiguous nucleic acids or amino acids. In yet further embodiments, the length of the sequence sharing identity is 50 or more contiguous nucleic acids or amino acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous nucleic acids or amino acids.

As set forth herein, nucleic acid variants such as codon-optimized variants encoding FVIII or hFVIII-BDD will be distinct from wild-type but may exhibit sequence identity with wild-type FVIII protein with, or without B-domain. In codon-optimized nucleic acid variants encoding FVIII or hFVIII-BDD, at the nucleotide sequence level, a codon-optimized nucleic acid encoding FVIII or hFVIII-BDD will typically be at least about 70% identical, more typically about 75% identical, even more typically about 80%-85% identical to wild-type FVIII encoding nucleic acid. Thus, for example, a codon-optimized nucleic acid encoding FVIII or hFVIII-BDD may have 75%-85% identity to wild-type FVIII encoding gene, or to each other.

At the amino acid sequence level, a variant such as a variant FVIII or hFVIII-BDD protein will be at least about 70% identical, more typically about 75% identical, or 80% identical, even more typically about 85% identical, or 90% or more identical to the full-length human FVIII or hFVIII BDD amino acid sequence. In other embodiments, a variant such as a variant FVIII or hFVIII-BDD protein has at least 90%, 91%, 92%, 93%, 94%, 95%, 96%, 97%, 98%, 99% or more identity to a reference sequence, e.g. wild-type full-length FVIII protein or hFVIII BDD SQ.

To determine identity, if the FVIII (e.g., codon-optimized nucleic acid encoding FVIII) retains the B-domain, it is appropriate to compare identity to wild-type FVIII. If the FVIII (e.g., a codon-optimized nucleic acid encoding hFVIII-BDD) has a B-domain deletion, it is appropriate to compare identity to wild-type FVIII that also has a B-domain deletion.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information (found on the world wide web at ncbi nlm nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acid molecules, expression vectors (e.g., vector genomes), plasmids, including nucleic acids and nucleic acid variants encoding FVIII or hFVIII-BDD of the invention may be prepared by using recombinant DNA technology methods. The availability of nucleotide sequence information enables preparation of isolated nucleic acid molecules of the invention by a variety of means. For example, codon-optimized nucleic acid variants encoding FVIII or hFVIII-BDD can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Methods and uses of the invention of the invention include delivering (transducing) nucleic acid (transgene) into host cells, including dividing and/or non-dividing cells. The nucleic acids, rAAV vector, methods, uses and pharmaceutical formulations of the invention are additionally useful in a method of delivering, administering or providing a FVIII or hFVIII-BDD to a subject in need thereof, as a method of treatment. In this manner, the nucleic acid is transcribed and the protein may be produced in vivo in a subject. The subject may benefit from or be in need of the FVIII or hFVIII-BDD because the subject has a deficiency of FVIII, or because production of FVIII in the subject may impart some therapeutic effect, as a method of treatment or otherwise.

rAAV vectors comprising a genome with a nucleic acid or nucleic acid variant encoding FVIII or hFVIII-BDD permit the treatment of genetic diseases, e.g., a FVIII deficiency. For deficiency state diseases, gene transfer can be used to bring a normal gene into affected tissues for replacement therapy.

In particular embodiments, rAAV vectors comprising a genome with a nucleic acid or nucleic acid variant encoding FVIII or hFVIII-BDD may be used, for example, as therapeutic and/or prophylactic agents (protein or nucleic acid) which modulate the blood coagulation cascade or as a transgene in gene. For example, an encoded FVIII or hFVIII-BDD may have similar coagulation activity as wild-type FVIII, or altered coagulation activity compared to wild-type FVIII. Gene therapy strategies allow continuous expression of FVIII or hFVIII-BDD in hemophilia A patients.

Administration of FVIII or hFVIII-BDD-encoding rAAV vectors to a patient results in the expression of FVIII or hFVIII-BDD protein which serves to alter the coagulation cascade. In accordance with the invention, expression of FVIII or hFVIII-BDD protein as described herein, or a functional fragment, increases hemostasis.

“Adeno-associated viruses” (AAV) are in the parvovirus family. AAV are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, rAAV vectors are able to deliver heterologous polynucleotide sequences (e.g., therapeutic proteins and agents) to human patients without causing substantial AAV pathogenesis or disease. rAAV vectors possess a number of desirable features for such applications, including tropism for dividing and non-dividing cells. These vector systems have been tested in humans targeting retinal epithelium, liver, skeletal muscle, airways, brain, joints and hematopoietic stem cells.

AAV vectors do not typically include viral genes associated with pathogenesis. Such vectors typically have one or more of the wild type AAV genes deleted in whole or in part, for example, rep and/or cap genes, but retain at least one functional flanking ITR sequence, as necessary for the rescue, replication, and packaging of the recombinant vector into an AAV vector particle. For example, only the essential parts of vector e.g., the ITR elements, respectively are included. An AAV vector genome would therefore include sequences required in cis for replication and packaging (e.g., functional ITR sequences)

Recombinant AAV vector, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, -rh74, -rh10, hu37 or AAV-2i8, for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be a AAV1, AAV3, AAV4, AAV5, AAV6, AAV7, AAV8, AAV9, AAV10, AAV11, AAV12, Rh10, hu37, Rh74 or AAV-2i8 or variant thereof, for example.

One embodiment provides the use of a replication deficient adeno-associated virus (AAV) to deliver a human FVIII (hFVIII or hF8) gene to liver cells of patients (human subjects) diagnosed with hemophilia A. In this embodiment, the recombinant AAV vector (rAAV) used for delivering the hFVIII gene (“rAAV.hFVIII”) should have a tropism for the liver (e.g., a rAAV bearing an AAVhu.37 or an AAVrh.10 capsid), and the hFVIII transgene should be controlled by liver-specific expression control elements. In one embodiment, the expression control elements include one or more of the following: a transthyretin enhancer (enTTR); a transthyretin (TTR) promoter; and a polyA signal. In another embodiment, the expression control elements include one or more of the following: a shortened α1-microglogulin/bikunin precursor (ABPS) enhancer, and enTTR; a transthyretin (TTR) promoter; and a polyA signal. In one embodiment, the expression control elements include one or more of the following: a transthyretin enhancer (enTTR); an alpha 1 anti-trypsin (A1AT) promoter; and a polyA signal. In another embodiment, the expression control elements include one or more of the following: an ABPS enhancer, and enTTR; an A1AT promoter; and a polyA signal. Such elements are further described herein.

In one embodiment, the hFVIII gene encodes a B-domain deleted (BDD) form of factor VIII, in which the B-domain is replaced by a short amino acid linker (FVIII-BDD-SQ, also referred to herein as hFVIII). In one embodiment, the FVIII-BDD-SQ protein sequence is shown in SEQ ID NO: 3. In one embodiment, the FVIII-BDD-SQ coding sequence is shown in SEQ ID NO: 1. The coding sequence for hFVIII is, in one embodiment, codon optimized for expression in humans. Such sequence may share less than 80% identity to the native hFVIII coding sequence (SEQ ID NO: 1). In one embodiment, the hFVIII coding sequence is that shown in SEQ ID NO: 2. In particular embodiments, adeno-associated virus (AAV) vectors include AAV1, AAV2, AAV3, AAV4, AAV5, AAV6, AAV7, AAV8, AAV9, AAV10, AAV11, AAV12, Rh10, hu37, Rh74 and AAV-2i8, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670) and U.S. Pat. No. 9,169,299 to patentee Leland Stanford Junior University, which discloses LK01, LK02, LK03, etc.

AAV variants include variants and chimeras of AAV1, AAV2, AAV3, AAV4, AAV5, AAV6, AAV7, AAV8, AAV9, AAV10, AAV11, AAV12, Rh10, hu37, Rh74 and AAV-2i8 capsid. Accordingly, AAV vectors and AAV variants (e.g., capsid variants) that include (encapsidate or package) nucleic acid or nucleic acid variant encoding FVIII or hFVIII-BDD are within the scope of the vectors useful in the present inventions.

In various exemplary embodiments, an AAV vector related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, Rh10, Rh74, hu37 or AAV-2i8 (e.g., such as an ITR, or a VP1, VP2, and/or VP3 sequences).

In one embodiment of the invention, rAAV vector comprising a nucleic acid or variant encoding FVIII or hFVIII-BDD, may be administered to a patient via infusion in a biologically compatible carrier, for example, via intravenous injection. The rAAV vectors may optionally be encapsulated into liposomes or mixed with other phospholipids or micelles to increase stability of the molecule.

Accordingly, rAAV vectors and other compositions, agents, drugs, biologics (proteins) can be incorporated into pharmaceutical compositions. Such pharmaceutical compositions are useful for, among other things, administration and delivery to a subject in vivo or ex vivo.

In particular embodiments, pharmaceutical compositions also contain a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Pharmaceutical compositions and delivery systems appropriate for the compositions, methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

In one embodiment, the dose administered to a patient suffering from hemophilia A is selected from the group consisting of 0.5×10¹³, 0.6×10¹³, 0.7×10¹³, 0.8×10¹³, 0.9×10¹³, 1.0×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2.0×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3.0×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, and 4.0×10¹³ genome copies/kg, or is a dose of from 0.5×10¹³ to 4×10¹³ genome copies/kg, or is 0.5×10¹³, 1.0×10¹³, 2.0×10¹³, or 4.0×10¹³ genome copies/kg of an AAV gene therapy vector for delivering human FVIII or a variant thereof. In a further embodiment, the dosage is one of the amounts disclosed herein that is sufficient to obtain sustained human FVIII procoagulant activity as measured 10 months after administration, 20 months after administration, 30 months after administration, and/or 40 months after administration.

In further embodiments the dosage is sufficient to achieve a therapeutic effect by resulting in a FVIII activity that is greater than 1% of FVIII activity found in a normal individual, greater than or equal to 5% of FVIII activity found in normal individuals, or between 1-5% of FVIII activity found in normal individuals, which activity may be measured using a chromogenic assay or an aPTT assay. In further embodiments administration of one the dosage amounts disclosed herein changes a severe disease phenotype to a moderate one. A severe phenotype is characterized by joint damage and life-threatening bleeds.

In one embodiment, the dose administered to a patient suffering from hemophilia A is a minimally effective dose that is 3×10¹¹. In another embodiment, the therapeutically effective dosage is less than 4.0×10¹² genome copies/kg, optionally wherein 19 months after administration sustained human FVIII activity levels are achieved, such as sustained human FVIII activity levels of at least 1% or at least 5% of normal human FVIII activity levels, preferably between 5 and 10% of normal human FVIII activity levels.

In another embodiment the dose is selected from the group consisting of 1.5×10¹², 1.6×10¹², 1.7×10¹², 1.8×10¹², 1.9×10¹², 2.0×10¹², 2.1×10¹², 2.2×10¹², 2.3×10¹², 2.4×10¹², 2.5×10¹², 2.6×10¹², 2.7×10¹², 2.8×10¹², 2.9×10¹², 3.0×10¹², 3.1×10¹², 3.2×10¹², 3.3×10¹², 3.4×10¹², 3.5×10¹², 3.6×10¹², 3.7×10¹², 3.8×10¹², and 3.9×10¹² genome copies/kg. In a further embodiment, the dosage is selected from the group consisting of 1.5×10¹², 1.6×10¹², 1.7×10¹², 1.8×10¹², 1.9×10¹², 2.0×10¹², 2.1×10¹², 2.2×10¹², 2.3×10¹², 2.4×10¹², 2.5×10¹², 2.6×10¹², 2.7×10¹², 2.8×10¹², 2.9×10¹², 3.0×10¹², 3.1×10¹², 3.2×10¹², 3.3×10¹², 3.4×10¹², 3.5×10¹², 3.6×10¹², 3.7×10¹², 3.8×10¹², and 3.9×10¹² genome copies/kg and is sufficient to obtain sustained human FVIII procoagulant activity as measured 10 months after administration, 20 months after administration, 30 months after administration, and/or 40 months after administration. In further embodiments the dosage is selected from the group consisting of 1.5×10¹², 1.6×10¹², 1.7×10¹², 1.8×10¹², 1.9×10¹², 2.0×10¹², 2.1×10¹², 2.2×10¹², 2.3×10¹², 2.4×10¹², 2.5×10¹², 2.6×10¹², 2.7×10¹², 2.8×10¹², 2.9×10¹², 3.0×10¹², 3.1×10¹², 3.2×10¹², 3.3×10¹², 3.4×10¹², 3.5×10¹², 3.6×10¹², 3.7×10¹², 3.8×10¹², and 3.9×10¹² genome copies/kg and is sufficient to achieve a therapeutic effect by resulting in a FVIII activity that is greater than 1% of FVIII activity found in a normal individual, greater than or equal to 5% of FVIII activity found in normal individuals, or between 1-5% of FVIII activity found in normal individuals, which activity may be measured using a chromogenic assay or an aPTT assay.

FVIII levels in normal humans are about 150-200 ng/ml plasma, but may be less (e.g., range of about 100-150 ng/ml) or greater (e.g., range of about 200-300 ng/ml) and still considered normal due to functioning clotting as determined, for example, by an activated partial thromboplastin time (aPTT) one-stage clotting assay. Thus, a therapeutic effect can be achieved by expression of FVIII or hFVIII-BDD or a variant thereof such that the total amount of FVIII in the subject/human is greater than 1% of the FVIII present in normal subjects/humans, e.g., 1% of 100-300 ng/ml.

The embodiments described herein relate to an AAV gene therapy vector for delivering normal or functional human FVIII to a subject in need thereof, following intravenous administration of the vector resulting in long-term, perhaps 10 years or more, of clinically meaningful correction of the bleeding defect. The subject patient population is patients with moderate to severe hemophilia A. The inventive AAV vector treatment converts severe hemophilia A patients to either moderate or mild hemophilia A thus relieving such patients of the need to be on a prophylaxis regimen.

In certain embodiments, the effective amount or a sufficient amount of the AAV gene therapy vector is provided in a single administration on a single day, or in multiple administrations over a period of 1 to 60 days. In another embodiment, the effective amounts are delivered over two or more administrations spaced apart by at least ten years.

For HemA, an effective amount would be an amount that reduces frequency or severity of acute bleeding episodes in a subject, for example, or an amount that reduces clotting time as measured by a clotting assay, for example.

Methods and uses of the invention include delivery and administration systemically, regionally or locally, including, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720).

Subjects can be tested for one or more liver enzymes for an adverse response or to determine if such subjects are appropriate for treatment according to a method of the invention. Candidate hemophilia subjects can therefore be screened for amounts of one or more liver enzymes prior to treatment according to a method of the invention. Subjects also can be tested for amounts of one or more liver enzymes after treatment according to a method of the invention. Such treated subjects can be monitored after treatment for elevated liver enzymes, periodically, e.g., every 1-4 weeks or 1-6 months. Exemplary liver enzymes include alanine aminotransferase (ALT), aspartate aminotransferase (AST), and lactate dehydrogenase (LDH), but other enzymes indicative of liver damage can also be monitored. A normal level of these enzymes in the circulation is typically defined as a range that has an upper level, above which the enzyme level is considered elevated, and therefore indicative of liver damage. A normal range depends in part on the standards used by the clinical laboratory conducting the assay. In one embodiment, the liver enzymes are monitored after administration of the AAV gene therapy. In another embodiment, the dosages of the AAV gene therapy result in levels of ALT, AST, and LDH that are acceptable as understood by those of skill in the art, and in a further embodiment the liver enzymes are at levels at or below the levels of ALT, AST and LDH shown in the patient blood tests reported in the figures and examples herein. In one embodiment the methods provide effective therapy for treatment of hemophilia A without unacceptable elevation of ALT, AST, and/or LDH. In one embodiment the methods provide effective treatment for hemophilia A without elevation of ALT, AST, and/or LDH above 10%, 20%, 25%, 30%, 35%, 40% or 50% of the patient's enzyme level prior to administration of the AAV gene therapy vector.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, virus (e.g., AAV) vector, or virus particle and optionally a second active, such as another compound, agent, drug or composition. In one embodiment the kit comprises a first container containing the AAV vector and a second container containing a diluent. In one embodiment the kit comprises a first container comprising the AAV vector and a separate syringe or other medical device for use in administration of the AAV vector.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

All patents, patent applications, publications, and other references, GenBank citations and ATCC citations cited herein are hereby incorporated herein by reference in their entireties. In case of conflict, the specification, including definitions, will control.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

A “factor VIII antigen assay” preferably is an ELISA for FVIII antigen such as are commercially available. Another example of an ELISA assay is found in Sahud et al., “ELISA system for detection of immune responses to FVIII: A study of 246 samples and correlation with the Bethesda assay,” Haemophilia (2007) 13, 317-322. Another example of a suitable FVIII antigen assay is a Luminex® fluorescence immunoassay, such as the one described in Butenas et al., “The ‘normal’ factor VIII concentration in plasma,” Thromb Res. 2010 August; 126(2): 119-123.

A “chromogenic FVIII assay” may be performed using Coatest SP reagents (Chromogenix) such as the following assay: FVIII samples and a FVIII standard (e.g. purified wild-type rFVIII calibrated against the 7th international FVIII standard from NIBSC) are diluted in Coatest assay buffer (50 mM Tris, 150 mM NaCl, 1% BSA, pH 7.3, with preservative). Fifty μl of samples, standards, and buffer negative control are added to 96-well microtiter plates (Nunc) in duplicates. The factor IXa/factor X reagent, the phospholipid reagent and CaCl₂) from the Coatest SP kit are mixed 5:1:3 (vol:vol:vol) and 75 μl of this is added to the wells. After 15 min incubation at room temperature 50 μl of the factor Xa substrate S2765/thrombin inhibitor 1-2581 mix is added and the reaction is incubated 10 min at room temperature before 25 μl 1 M citric acid, pH 3, is added. The absorbance at 415 nm is measured on a Spectramax microtiter plate reader (Molecular Devices) with absorbance at 620 nm used as reference wavelength. The value for the negative control is subtracted from all samples and a calibration curve is prepared by linear regression of the absorbance values plotted vs. FVIII concentration. The specific activity is calculated by dividing the activity of the samples with the protein concentration determined by HPLC.

An “activated partial thromboplastin time assay” or “aPTT assay” is a one-stage assay based upon the activated partial thromboplastin time (aPTT). FVIII acts as a cofactor in the presence of Factor IXa, calcium, and phospholipid in the enzymatic conversion of Factor X to Xa. In this assay, the diluted test samples are incubated at 37° C. with a mixture of FVIII deficient plasma substrate and a PTT reagent. Calcium chloride is added to the incubated mixture and clotting is initiated. An inverse relationship exists between the time (seconds) it takes for a clot to form and logarithm of the concentration of FVIII:C. Activity levels for unknown samples are interpolated by comparing the clotting times of various dilutions of test material with a curve constructed from a series of dilutions of standard material of known activity and are reported in International Units per mL (IU/mL).

In one embodiment, the AAV vector is selected from AAVrh10 vectors that are designed to drive liver-specific expression of the codon-optimized BDD hFVIII gene within the size constraints of AAV3. Within a total genome size of <5,250 bp, 42 enhancer/promoter combinations of three shortened liver-specific promoters and up to three liver-specific enhancer sequences were evaluated and reported in U.S. Pat. No. 10,888,628. After evaluating hFVIII activity and immunogenicity of the transgene in a mouse model of hemophilia A (FVIII knockout “KO” mice), an additional capsid-specific immunogenicity evaluation was performed³. Based on these analyses, it was preferred to use AAVrh10 and AAVhu37 capsids and the E03.TTR and E12.A1AT enhancer/promoter combinations for the methods and dosages of the invention.

Vectors Suitable for Use in Gene Therapy

The following AAV vectors may be used in the methods of treatment disclosed herein.

The AAV particles comprising an AAV vector delivering a functional FVIII as described in U.S. Pat. No. 10,888,628 may be used, in one embodiment. The gene therapy vectors used in the present invention comprise a rAAV capsid carrying a viral genome that encodes FVIII, preferably human FVIII, or a variant thereof having FVIII procoagulant activity. The viral genome includes expression control elements. In one embodiment, the expression control elements include one or more of the following: a transthyretin enhancer (enTTR); a transthyretin (TTR) promoter; and a polyA signal. In another embodiment, the expression control elements include one or more of the following: a shortened α1-microglogulin/bikunin precursor (ABPS) enhancer, and enTTR; a transthyretin (TTR) promoter; and a polyA signal. In one embodiment, the expression control elements include one or more of the following: a transthyretin enhancer (enTTR); an alpha 1 anti-trypsin (A1AT) promoter; and a polyA signal. In another embodiment, the expression control elements include one or more of the following: an ABPS enhancer, and enTTR; an A1AT promoter; and a polyA signal. Such elements are further described herein.

In one embodiment, the hFVIII gene encodes a B-domain deleted (BDD) form of FVIII, in which the B-domain is replaced by a short amino acid linker (FVIII-BDD-SQ, also referred to herein as hFVIII). In one embodiment, the FVIII-BDD-SQ protein sequence is shown in SEQ ID NO: 3. In one embodiment, the FVIII-BDD-SQ coding sequence is shown in SEQ ID NO: 1. The coding sequence for hFVIII is, in one embodiment, codon optimized for expression in humans. Such sequence may share less than 80% identity to a wild-type hFVIII coding sequence or to the FVIII-BDD-SQ coding sequence of SEQ ID NO: 1. In one embodiment, the hFVIII coding sequence is that shown in SEQ ID NO: 2. In another embodiment, the vector genome encodes a polypeptide that when expressed has the procoagulant activity of human FVIII, such as at least 50% of the activity of wild type human FVIII as measured in an aPPT assay or a chromogenic FVIII assay. In another embodiment, the vector genome comprises a 5′ inverted terminal repeat (ITR) sequence, preferably as shown in SEQ ID NO:11, the transthyretin (TTR) promoter, the TTR enhancer (enTTR), preferably as shown in SEQ ID NO:5, codon optimized human coagulation factor VIII (FVIII) cDNA, the artificial polyadenylation signal, preferably as shown in SEQ ID NO:10, and the 3′ ITR, preferably as shown in SEQ ID NO:12, and the capsid comprises AAVhu.37 capsid, preferably having the amino acid sequence of GenBank, accession: AAS99285, shown in SEQ ID NO:17, or a sequence with at least 80% identity to SEQ ID NO:17, or the capsid comprises AAVrh10 capsid, preferably having the amino acid sequence of GenBank, accession: AA088201, shown in SEQ ID NO:18 or a sequence with at least 80% identity to SEQ ID NO:18. In a further embodiment, a recombinant AAV comprises an AAV capsid and a vector genome packaged therein, the AAV vector genome substantially comprising or consisting of nucleic acid sequences for the 5′ inverted terminal repeat (ITR) sequence shown in SEQ ID NO:11, the transthyretin (TTR) promoter as shown in SEQ ID NO:7, the TTR enhancer (enTTR) shown in SEQ ID NO:5, codon optimized human coagulation factor VIII (FVIII) cDNA, the artificial polyadenylation signal shown in SEQ ID NO:10, the 3′ ITR shown in SEQ ID NO:12, and the capsid comprises AAVhu.37 capsid having the amino acid sequence of GenBank accession number AAS99285, shown in SEQ ID NO:17. In one embodiment the AAV gene therapy vector is BAY 2599023 (AAVhu37.hFVIIIco).

Other useful AAV for the present invention include any of the following:

A recombinant adeno-associated virus (rAAV) serotype 2 comprising wild-type AAV2 viral capsid, and a nucleic acid encoding a human FVIII sequence in which the B domain is replaced with the 14 amino acid SQ sequence, and still more preferably with a codon-optimized human FVIII sequence in which the B domain is replaced with the 14 amino acid SQ sequence; preferably in which the viral genome further comprises the following expression elements described in U.S. Pat. Application Pub. No. 20150071883: an AAV2 5′ inverted terminal repeat (ITR) sequence, a 34 base human apolipoprotein E (ApoE)/C1 enhancer, a 32 base human alpha anti-trypsin (AAT) promoter distal X region, a 186 base human AAT promoter, including 42 bases of 5′ untranslated region (UTR) sequence, a 49 base synthetic polyadenylation sequence, and an AAV2 3′ ITR sequence. In one embodiment, the rAAV is any one of the rAAV vectors described in U.S. Pat. Application Pub. No. US 20150071883; in a further embodiment, the vector is Valoctocogene roxaparvovec.

In one embodiment, the rAAV vector is Giroctocogene fitelparvovec rAAV6 vector.

In one embodiment, the vector is the rAAV vector described in U.S. Pat. Application Pub. No. 20170119906.

In one embodiment, the vector is the rAAV gene therapy vector described in U.S. Pat. Application Pub. No. 20200237930.

In one embodiment, the vector is the rAAV gene therapy vector described in U.S. Pat. Application Pub. No. 20190240350.

In one embodiment, the vector is a vector disclosed in U.S. Pat. Application Pub. No. 20200237930, such as SPK-8011 rAAV-LK03.

In one embodiment, the vector is the rAAV vector described in U.S. Pat. Application Pub. No. 20180312571.

Certain embodiments described in the application relate to the use of a replication deficient adeno-associated virus (AAV) to deliver a human Factor VIII (hFVIII) gene to liver cells of patients (human subjects) diagnosed with HA. For these embodiments, the recombinant AAV vector (rAAV) used for delivering the hFVIII gene (“rAAV.hFVIII”) should have a tropism for the liver (e.g., an rAAV bearing an AAVhu.37 or AAVrh.10 capsid), and the hFVIII transgene should be controlled by liver-specific expression control elements. In one embodiment, the expression control elements include one or more of the following: a transthyretin (TTR) enhancer; a transthyretin (TTR) promoter; and a polyA signal. Such elements are further described herein.

As used herein, “AAVhu.37 capsid” refers to the hu.37 having the amino acid sequence of GenBank accession number AAS99285, which is SEQ ID NO: 17. Some variation from this encoded sequence is permitted, which may include sequences having about 99% identity to the referenced amino acid sequence in AAS99285 and U.S. Pat. Application Pub. No. 2015/0315612 (i.e., less than about 1% variation from the referenced sequence). Methods of generating the capsid, coding sequences therefore, and methods for production of rAAV viral vectors have been described. See, e.g., Gao et al., Proc. Natl. Acad. Sci. U.S.A. 100 (10), 6081-6086 (2003) and US Pat. Application Pub. No. 2015/0315612.

As used herein, “AAVrh10 capsid” refers to the rh.10 having the amino acid sequence of GenBank accession number AA088201, which is SEQ ID NO: 18. Some variation from this encoded sequence is permitted, which may include sequences having about 99% identity to the referenced amino acid sequence in AA088201 and U.S. Pat. Application Pub. No. 2013/0045186A1 (i.e., less than about 1% variation from the referenced sequence), preferably a sequence variation such that the integrity of the ligand-binding site for the affinity capture purification is maintained and the change in sequences does not substantially alter the pH range for the capsid for the ion exchange resin purification. Methods of generating the capsid, coding sequences therefore, and methods for production of rAAV viral vectors have been described. See, e.g., Gao et al., Proc. Natl. Acad. Sci. U.S.A. 100 (10), 6081-6086 (2003) and U.S. Pat. Application Pub. No. 2013/0045186A1.

A preferred AAV for use in the methods of the present disclosure is one that has one or more of these features, numbered as different embodiments:

1. The AAV comprises an AAV capsid and a vector genome packaged therein, said vector genome comprising:

an AAV 5′-inverted terminal repeat (ITR) sequence;

a liver-specific promoter;

a coding sequence encoding a human Factor VIII having coagulation function; and

an AAV 3′-ITR sequence,

preferably wherein said coding sequence comprises the nucleotide sequence as set forth in SEQ ID NO: 2.

2. The AAV capsid is a hu.37 capsid.

3. The rAAV according to embodiment 1, wherein the AAV 5′-ITR is from AAV2.

4. The rAAV according to embodiment 3, wherein the AAV 5′-ITR comprises the nucleotide sequence as set forth in SEQ ID NO: 11.

5. The rAAV according to embodiment 1, wherein the AAV 3′-ITR is from AAV2.

6. The rAAV according to embodiment 5, wherein the AAV 3′-ITR comprises the nucleotide sequence as set forth in SEQ ID NO: 12.

7. The rAAV according to embodiment 1, wherein the AAV 5′-ITR and AAV 3′-ITR are from AAV2.

8. The rAAV according to embodiment 1, wherein the liver-specific promoter is a transthyretin (TTR) promoter.

9. The rAAV according to embodiment 8, wherein the TTR promoter comprises the nucleotide sequence as set forth in SEQ ID NO: 7.

10. The rAAV according to embodiment 1, wherein the vector genome further comprises an enhancer.

11. The rAAV according to embodiment 10, wherein the enhancer is a liver-specific enhancer.

12. The rAAV according to embodiment 11, wherein the liver-specific enhancer is a transthyretin enhancer (enTTR).

13. The rAAV according to embodiment 12, wherein the enTTR comprises the nucleotide sequence as set forth in SEQ ID NO: 5.

14. The rAAV according to embodiment 1, wherein the vector genome further comprises a polyA sequence.

15. The rAAV according to embodiment 14, wherein the polyA sequence comprises the nucleotide sequence as set forth in SEQ ID NO: 10.

16. The rAAV according to embodiment 1, wherein the vector genome is 5 kilobases to 5.5 kilobases in size.

17. A recombinant adeno-associated virus (rAAV) comprising an AAVhu.37 capsid and a vector genome packaged therein, said vector genome comprising:

an AAV 5′-inverted terminal repeat (ITR) sequence;

a transthyretin (TTR) promoter;

transthyretin enhancer (enTTR);

a coding sequence encoding a human Factor VIII having coagulation function; and

an AAV 3′-ITR sequence,

preferably wherein said coding sequence comprises the nucleic acid sequence set forth in SEQ ID NO: 2.

In one embodiment, the hFVIII gene encodes the hFVIII protein shown in SEQ ID NO: 3, which is a FVIII in which the B domain is deleted (BDD) and replaced by a short 14 amino acid linker (FVIII-BDD-SQ). Thus, in one embodiment, the hFVIII transgene can include, but is not limited to, one or more of the sequences provided by SEQ ID NO:1 or SEQ ID NO: 2. SEQ ID NO: 1 provides the cDNA for human FVIII-BDD-SQ. SEQ ID NO: 2 provides an engineered cDNA for human FVIII-BDD-SQ, which has been codon optimized for expression in humans (sometimes referred to herein as hFVIIIco-SQ or hFVIIIco-BDD-SQ). It is to be understood that reference to hFVIII herein may, in some embodiments, refer to the hFVIII-BDD-SQ native or codon optimized sequence. Alternatively or additionally, web-based or commercially available computer programs, as well as service based companies may be used to back translate the amino acid sequences to nucleic acid coding sequences, including both RNA and/or cDNA. See, e.g., backtranseq by EMBOSS, www.ebi.ac.uk/Tools/st/; Gene Infinity (www.geneinfinity.org/sms-/smsbacktranslation.html); ExPasy (www.expasy.org/tools/). It is intended that all nucleic acids encoding the described hFVIII polypeptide sequences are encompassed, including nucleic acid sequences which have been optimized for expression in the desired target subject (e.g., by codon optimization). In one embodiment, the nucleic acid sequence encoding hFVIII shares at least 95% identity with the hFVIII coding sequence of SEQ ID NO: 1. In another embodiment, the nucleic acid sequence encoding hFVIII shares at least 90, 85, 80, 75, 70, or 65% identity with the hFVIII coding sequence of SEQ ID NO: 1. In one embodiment, the nucleic acid sequence encoding hFVIII shares about 77% identity with the hFVIII coding sequence of SEQ ID NO: 1. In one embodiment, the nucleic acid sequence encoding hFVIII is SEQ ID NO: 2. In another embodiment, the nucleic acid sequence encoding hFVIII shares at least 99%, 97%, 95%, 90%, 85%, 80%, 75%, 70%, or 65% identity with the hFVIII coding sequence of SEQ ID NO: 1 or SEQ ID NO: 2. In another embodiment, the nucleic acid sequence encoding hFVIII is SEQ ID NO: 19. In another embodiment, the nucleic acid sequence encoding hFVIII shares at least 90, 85, 80, 75, 70, or 65% identity with the hFVIII coding sequence of SEQ ID NO: 19. In yet another embodiment, the nucleic acid sequence encoding hFVIII shares at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity with the hFVIII coding sequence of SEQ ID NO: 1 or SEQ ID NO: 2. In yet another embodiment, the nucleic acid sequence encoding hFVIII shares at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity with the hFVIII coding sequence of SEQ ID NO: 19. See, Ward et al, Codon optimization of human factor VIII cDNAs leads to high-level expression, Blood, 117(3):798-807 (January 2011), for a discussion of various variants of FVIII-SQ, including codon optimized variants.

In one embodiment, the vector genome is nt 1 to nt 5110 of SEQ ID NO: 13. In one embodiment, the vector genome is nt 1 to nt 5194 of SEQ ID NO: 14. In one embodiment, the vector genome is nt 1 to nt 5138 of SEQ ID NO: 15. In another embodiment, the vector genome is nt 1 to nt 5222 of SEQ ID NO: 16.

In one embodiment, the AAVhu37 vector is provided in a pharmaceutical composition Which comprises an aqueous carrier; excipient, diluent or buffer. In one embodiment, the buffer is PBS. In a specific embodiment, the AAVhu37 formulation is a suspension containing an effective amount of AAVhu37 vector suspended in an aqueous solution containing 0.001% Pluronic F-68 in TMN200 (200 mM sodium chloride, 1 mM magnesium chloride, 20 (mM Tris, pH 8.0). However, various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

The inventive methods of the present disclosure include methods that result in an increase in FVIII activity to 3% of normal from baseline up to 52 weeks after administration of the gene therapy treatment. In one embodiment, patients achieve desired circulating Mil levels of 5% or greater of normal human FVIII activity after treatment with AAV gene therapy. In another embodiment, patients achieve circulating FVIII levels of 10%, 15%, 20% or greater of normal human FVIII activity after treatment with one of the AAV gene therapy methods described herein.

In another embodiment, the composition is readministered at a later date. Optionally, more than one readministration is permitted. In another embodiment, the vector is readministered about 5 years or more after the first administration. In another embodiment, the vector is readministered about 10 years or more after the first administration.

The viral vector dosages described herein may be used in preparing a medicament for delivering hFVIII to a subject (e.g., a human patient) in need thereof, supplying functional hFVIII to a subject, and/or for treating hemophilia A disease.

### EXAMPLES

### Example 1: Determining the Minimally Effective Dose of a Clinical Candidate AAV Vector in a Mouse Model of Hemophilia A

Introduction

The major limitation for an AAV-based gene therapy approach for hemophilia A is the size of the hFVIII coding sequence. The native hFVIII protein is a large, multi-domain glycoprotein with complementary DNA (cDNA) that exceeds the packaging capacity for recombinant AAV (>7 kb) (Grieger J C, Samulski R J, “Packaging capacity of adeno-associated virus serotypes: impact of larger genomes on infectivity and postentry steps,”. J Virol 2005; 79:9933-9944; Hasbrouck N C, High K A, “AAV-mediated gene transfer for the treatment of hemophilia B: problems and prospects.,” Gene Ther 2008; 15:870-875). As discussed in U.S. Pat. No. 10,888,628, the promoter and enhancer in the expression cassette have been extensively engineered (Greig J A et al., “Characterization of Adeno-Associated Viral Vector-Mediated Human Factor VIII Gene Therapy in Hemophilia A Mice.,” Hum Gene Ther 2017; 28:392-402) to complement the reduction in size of the hFVIII cDNA performed previously (also referred to as F8) (Ward N J et al., “Codon optimization of human factor VIII cDNAs leads to high-level expression,” Blood 2011; 117:798-807). The protein-replacement therapy drug—ReFacto® (Pfizer, New York, N.Y.)—was successfully designed to mimic the smallest active form of hFVIII by replacing the B domain with a 14-amino acid SQ linker (Sandberg H et al., “Structural and functional characteristics of the B-domain-deleted recombinant factor VIII protein, r-VIII SQ,” Thromb Haemost 2001; 85:93-100). Subsequent codon optimization of this B-domain-deleted (BDD) hFVIII-SQ (hFVIIIco-SQ) resulted in efficient packaging and increased expression from lentiviral vectors (Ward N J et al. Blood 2011; supra; Radcliffe P A et al.,” Analysis of factor VIII mediated suppression of lentiviral vector titres,” Gene Ther 2008; 15:289-297) and AAV (Greig J A et al. Hum Gene Ther 2017; supra; McIntosh J et al. Blood 2013; supra).

As discussed in U.S. Pat. No. 10,888,628, AAVrh10 vectors that are designed to drive liver-specific expression of the codon-optimized BDD hFVIII gene within the size constraints of AAV have been evaluated (Greig J A et al. Hum Gene Ther 2017; supra). Within a total genome size of <5,250 bp, 42 enhancer/promoter combinations of three shortened liver-specific promoters and up to three liver-specific enhancer sequences were generated. The hFVIII activity and immunogenicity of the transgene in a mouse model of hemophilia A (FVIII knockout [KO] mice) were evaluated and an additional capsid-specific immunogenicity evaluation was performed (Greig J A et al. Hum Gene Ther 2017; supra). Based on the resulting analyses, AAVrh10 and AAVhu37 capsids and the E03.TTR and E12.A1AT enhancer/promoter combinations were selected for further evaluation in nonhuman primates (Greig J A et al., “Optimized Adeno-Associated Viral-Mediated Human Factor VIII Gene Therapy in Cynomolgus Macaques,” Hum. Gene. Ther., published online Dec. 13, 2018).

Upon systemically administering 1.2×10¹³ genome copies (GC)/kg of AAVhu37.E03.TTR.hFVIIIco-SQ.PA75 (AAVhu37 capsid with transthyretin [TTR] enhancer [E03] and promoter and a codon-optimized version of the hFVIII protein, where the B domain was deleted and replaced by a short 14-amino acid linker [hFVIIIco-SQ]) to cynomolgus macaques, we obtained peak hFVIII activity levels of 23.4% of normal (Greig J A et al., Hum Gene Ther 2018, supra). While the majority of macaques (18/20) developed anti-hFVIII antibodies within 30 weeks of vector administration, two macaques administered with AAVhu37.E03.TTR.hFVIIIco-SQ.PA75 did not develop anti-hFVIII antibodies during the initial phase of the study (up to 30 weeks post-vector administration). These activity levels suggested that the AAVhu37-based gene therapy approach would be sufficient to modify severe hemophilia A phenotypes. The present disclosure describes a pharmacology study with safety measurements for the clinical candidate vector, AAVhu37.E03.TTR.hFVIIIco-SQ.PA75, in FVIII KO mice to determine the minimally effective dose (MED) and to support the initiation of a Phase 1 clinical trial in patients with hemophilia A.

Materials and Methods

AAV Vector Production

AAV vectors for research studies were produced by the Penn Vector Core at the University of Pennsylvania, as described previously (Gao G et al., “Biology of AAV serotype vectors in liver-directed gene transfer to nonhuman primates,” Mol Ther 2006; 13:77-87). Briefly, plasmids expressing hFVIIIco-SQ from EnTTR.TTR (E03.TTR) were packaged in the AAVhu37 capsid. Dimension Therapeutics (now Ultragenyx Gene Therapy, Novato, Calif.) produced the vector for the minimally effective dose (MED) study.

Mice

The FVIII KO mice (B6; 129S-F8tm1Kaz/J) were obtained from The Jackson Laboratory (Bar Harbor, Me.). A colony was maintained under specific pathogen-free conditions; the mice used for the pilot dose-ranging study were derived from this colony. Each cohort included ten animals. Prior to the study, it was determined that five animals per cohort is the minimal number to enable statistical analysis of study outcome; five additional animals per time point were included to ensure that enough study animals would be available for meaningful analysis in the occurrence of unexpected deaths, antibody generation to the hFVIII transgene, or other unanticipated events.

Pilot Dose-Ranging Study

Male FVIII KO aged 6-12 weeks received an intravenous (IV) injection with 1.5×10¹⁰, 1.5×10¹¹, 5×10¹¹, 1.5×10¹², 5×10¹², or 1.5×10¹³ GC/kg of AAVhu37.E03.TTR.hFVIIIco-SQ.PA75 via the tail vein. Vector was diluted in phosphate-buffered saline (PBS). The vehicle control group received an IV injection of 100 μl of PBS. Plasma was collected on days 7, 14, and 28 by retro-orbital bleeds into sodium citrate collection tubes. Mice were necropsied on day 28.

MED Study

Mice were obtained from Jackson Laboratories and housed five animals per cage in disposable micro-isolator mouse caging with corn cob bedding. Nestlets were provided for enrichment (Innovive, San Diego, Calif.). Certified irradiated Laboratory Rodent Diet 5002 (LabDiet, St. Louis, Mo.) was provided ad libitum. All interventions were performed during the light cycle. Mice were not fasted prior to blood collection.

In this study, male FVIII KO mice (n=100) aged 8-14 weeks, weighing 18.7-28.8 g in body weight were used. Prior to dosing, mice were first allocated to groups. Mice in the same cage belonged to the same group (mixing male mice from different cages into a new cage would cause fighting and possible death as these are hemophilic mice). Each group was randomly assigned to one of the dosing groups using an online program (Research Randomizer, http://www.randomizer.org/form.htm).

FVIII KO mice received an IV injection with 3×10¹¹, 1×10¹², 3×10¹², or 1×1013 GC/kg of AAVhu37.E03.TTR.hFVIIIco-SQ.PA75 via the tail vein. Vector was diluted in 0.01% (w/v) Pluronic F-68, 20 mM Tris, 200 mM NaCl, and 1 mM MgCl2 (pH 8.0±0.2). The control group that received an IV injection of 100 μl vehicle buffer contained no vector. Each cohort included ten animals. Prior to the study, five animals per cohort was determined to be the minimal number to enable statistical analysis of study outcome; five additional animals per time point were included to ensure that enough study animals would be available for meaningful analysis in the occurrence of unexpected deaths, antibody generation to the hFVIII transgene, or other unanticipated events.

Plasma was collected on days 7, 14, 28, and 56 via retro-orbital bleeds into sodium citrate collection tubes. Mice were necropsied on days 28 and 56. As the expressed transgene is human FVIII, most mice would likely have mounted an immune response to the transgene by day 56, neutralizing the activity of any further expressed transgene. Therefore, continuing the study past day 56 was not expected to yield additional pharmacological data.

Clinical Chemistries

Blood was collected at the time of necropsy by cardiac puncture in labeled serum gel separator brown-top tubes. After allowing the blood to clot for at least 30 minutes at room temperature, the samples were then centrifuged at 3,500×g for 5 minutes at room temperature. The serum was separated and then shipped to Antech GLP (Morrisville, N.C.) for analysis of alanine aminotransferase (ALT), aspartate aminotransferase (AST), alkaline phosphatase (ALP), gamma glutamyl transferase (GGT), total bilirubin, direct bilirubin, and total protein.

hFVIII Activity

hFVIII activity in plasma was measured using the Chromogenix Coatest® SP4 kit, according to the manufacturer's protocol (DiaPharma, West Chester, Ohio) (Greig J A et al., Hum Gene Ther 2017; supra). Briefly, this kit works by combining mouse plasma with unknown hFVIII levels with calcium, phospholipids, factors IXa and X. The rate of activation of factor X to Xa is dependent on the levels of hFVIII in the plasma sample. A standard curve was generated using known concentrations of BDD-hFVIII-SQ (XYNTHA antihemophiliac factor (recombinant), Wyeth Pharmaceuticals Inc., Dallas, Tex., USA).

Anti-hFVIII Immunoglobulin G

Immunoglobulin G (IgG) antibodies against hFVIII in mouse plasma were detected at the time of necropsy using the enzyme-linked immunosorbent assay (ELISA) as described previously (Greig J A et al., Hum Gene Ther 2017; supra). Plasma samples were diluted 1/100 or more and values that were five-fold over background levels (naïve mouse samples) were considered positive. The data were reported as anti-hFVIII IgG titers. Negative values are denoted as a titer of 1/50 to enable them to be visualized.

Histopathology

Tissues were harvested at necropsy for comprehensive histopathological examination. These tissues included the injection site, right testis, brain, liver, right kidney, lung, heart, and spleen. Tissues were fixed using 10% neutral buffered formalin, paraffin embedded, sectioned, and stained for histopathology using H&E stain. An experienced, board-certified veterinary pathologist evaluated liver sections in a blinded manner using scoring criteria. Histopathology slides for other tissues were evaluated and peer-reviewed for the highest vector dose group and the vehicle control group. If any findings were reported in the highest dose group, the next lower dose group was evaluated, and so on.

Vector Biodistribution

At the time of necropsy, liver was collected for biodistribution, frozen on dry ice, and stored at ≤−60° C. We extracted DNA and RNA from liver samples and performed quantitative polymerase chain reactions (qPCR) as described previously (Greig J A et al., Hum Gene Ther 2017; supra). Using qPCR targeting a vector-specific sequence, DNA and RNA samples were assayed for vector GC and vector-derived hFVIII transgene levels, respectively. Assay results were reported as GC per microgram of DNA (GC/μg). The vector GC per diploid genome were then calculated, assuming that one μg of DNA contains ˜2×10⁵ diploid genomes (Baumer C et al., “Exploring DNA quality of single cells for genome analysis with simultaneous whole-genome amplification,” Sci Rep 2018; 8:7476).

Statistical Analyses

The group average and standard error of the mean (SEM) were calculated and reported for the following: ALT, AST, total bilirubin, total protein, hFVIII activity, vector GCs, and hFVIII RNA transcript levels. Groups administered with vector and the vehicle control were compared using a Wilcoxon rank-sum test at each time point for ALT, AST, total protein, total bilirubin, hFVIII activity, vector GCs, and hFVIII RNA transcript levels (non-parametric evaluation as the data appeared to be non-normally distributed). Vector-administered groups were compared to each other using a two-sample Wilcoxon rank-sum test at each time point for hFVIII activity, vector GCs, and hFVIII RNA transcript levels. Linear mixed-effect modeling was used to compare the overall change between the vector- and vehicle control-administered groups across all time points. No multiple testing adjustment was performed. A p value of <0.05 was considered significant.

Results

Pilot Dose-Ranging Study in FVIII KO Mice

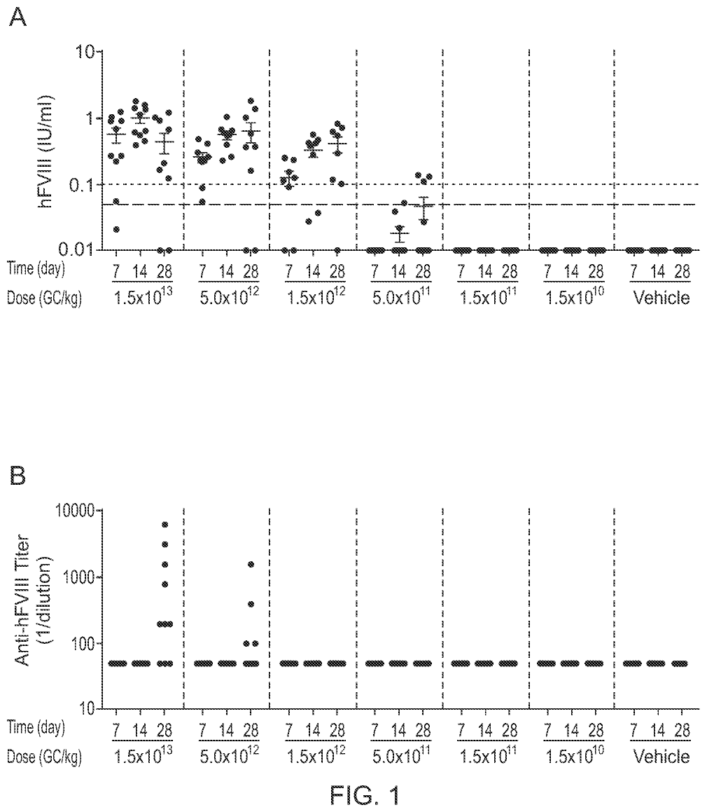

Based on the results of previous studies (Greig J A et al., Hum Gene Ther 2017; supra; Greig J A et al., Hum Gene Ther 2018, supra), the clinical candidate vector, AAVhu37.E03.TTR.hFVIIIco-SQ.PA75 was selected. Packaged within the AAVhu37 capsid, this vector had a transthyretin (TTR) enhancer (E03), TTR promoter, and codon-optimized version of the hFVIII protein, in which the B domain was deleted and replaced by a short, 14-amino acid linker (hFVIIIco-SQ). A pilot dose-ranging study in FVIII KO mice to determine the approximate MED was performed (FIG. 1). The FVIII KO mouse is a disease model for hemophilia A. As hemophilia A is an X-linked disease, male FVIII KO mice were used.

Male FVIII KO mice were injected intravenously (IV) at 6-12 weeks of age with AAVhu37.E03.TTR.hFVIIIco-SQ.PA75 at doses ranging from 1.5×10¹⁰ GC/kg to 1.5×10¹³ GC/kg or with vehicle. Human FVIII activity levels and anti-hFVIII IgG titers were measured in plasma samples taken throughout the in-life phase of the study and at the time of necropsy. Following vector administration, a dose-dependent increase in hFVIII activity levels, with no hFVIII activity detected in plasma at doses lower than 5.0×10¹¹ GC/kg was detected (FIG. 1A). Anti-hFVIII antibodies were only present in mice injected with 5.0×10¹² or 1.5×10¹³ GC/kg at day 28 (FIG. 1B). Therefore, the MED of AAVhu37.E03.TTR.hFVIIIco-SQ.PA75 in this research study was 5×10¹¹ GC/kg.

MED Study Rationale and Design

Male FVIII mice aged 8-14 weeks received an IV tail vein injection of vehicle control or AAVhu37.E03.TTR.hFVIIIco-SQ.PA75 at one of four doses: 3×10¹¹, 1×10¹², 3×10¹², or 1×10¹³ GC/kg. Necropsies were performed on mice 28 and 56 days after administration to capture peak and longer-term hFVIII activity.

Clinical Findings

During the study, two mice from the vehicle control group were euthanized for humane reasons (on days 15 and 24). In both cases, a full necropsy was performed and tissues were collected for analysis. Histopathology findings pointed to evidence of blood loss in conjunction with the clinical signs, although no direct evidence of hemorrhage or blood loss was observed. During the in-life phase of the study, we recorded clinical observations for 18 out of the 100 mice enrolled in this study that did not affect study outcome. An additional eight mice required supportive care.

Dose-Dependent Increase in hFVIII Activity

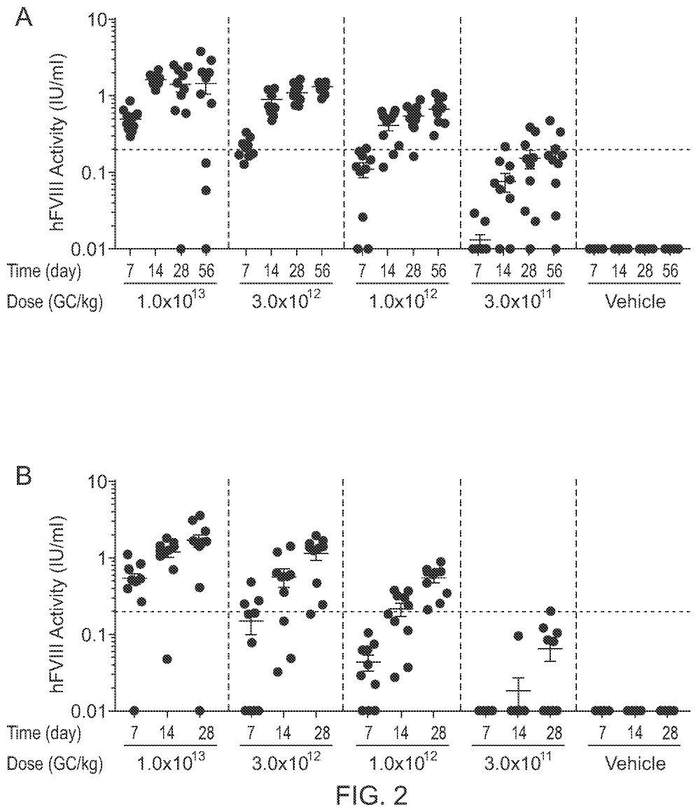

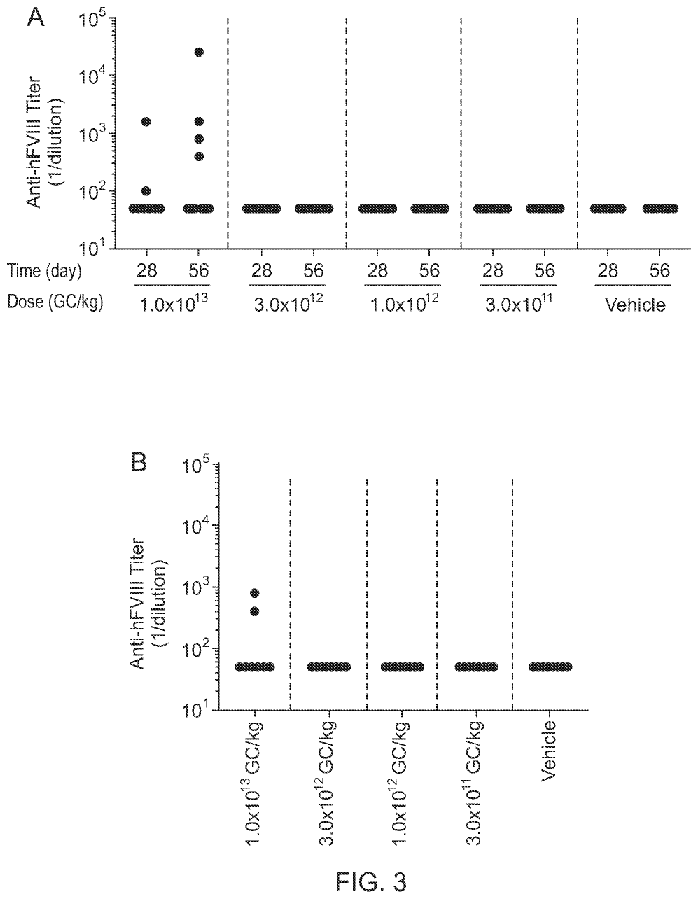

Plasma hFVIII activity levels were analyzed throughout the in-life phase of the study. As expected, hFVIII activity levels displayed a dose-dependent increase following IV administration of increasing vector doses (FIG. 2). hFVIII activity increased over the duration of the study from day 7 until the necropsy time point, unless anti-hFVIII IgG antibodies developed (FIG. 3).

For mice necropsied on day 56 post-vector administration, the average peak activity level in the high-dose group (1×10¹³ GC/kg) was 1.438 IU/ml (equivalent to 143.8% of normal FVIII levels) (FIG. 2A). By day 28 post-vector administration, anti-hFVIII IgG antibodies had developed in two out of the ten mice administered with the high dose, with an additional two mice in this group developing anti-hFVIII IgG antibodies by day 56 (FIG. 3A). All mice with detectable anti-hFVIII IgG antibodies exhibited a reduction in their individual hFVIII activity levels (FIGS. 2 and 3).

For mice necropsied on day 28 post-vector administration, the average peak activity level in the high-dose group (1×10¹³ GC/kg) was 1.684 IU/ml (FIG. 2B). Similar to mice necropsied at day 56 (FIG. 3A), anti-hFVIII IgG antibodies had developed in two mice in the only high-dose group by day 28 post-vector administration, which resulted in a decline in their individual hFVIII activity levels (FIG. 3B).

At the lowest dose evaluated in this study (3×10¹¹ GC/kg), the average peak activity level was 0.173 IU/ml at day 56 (FIG. 2A). hFVIII activity was detected at all doses of AAVhu37.E03.TTR.hFVIIIco-SQ.PA75 administered in both the day 28 and 56 cohorts; as a result, the MED is equal to 3×10¹¹ GC/kg (the lowest dose administered in this study).

No correlation was found between vector dose and elevations in serum ALT or AST levels.

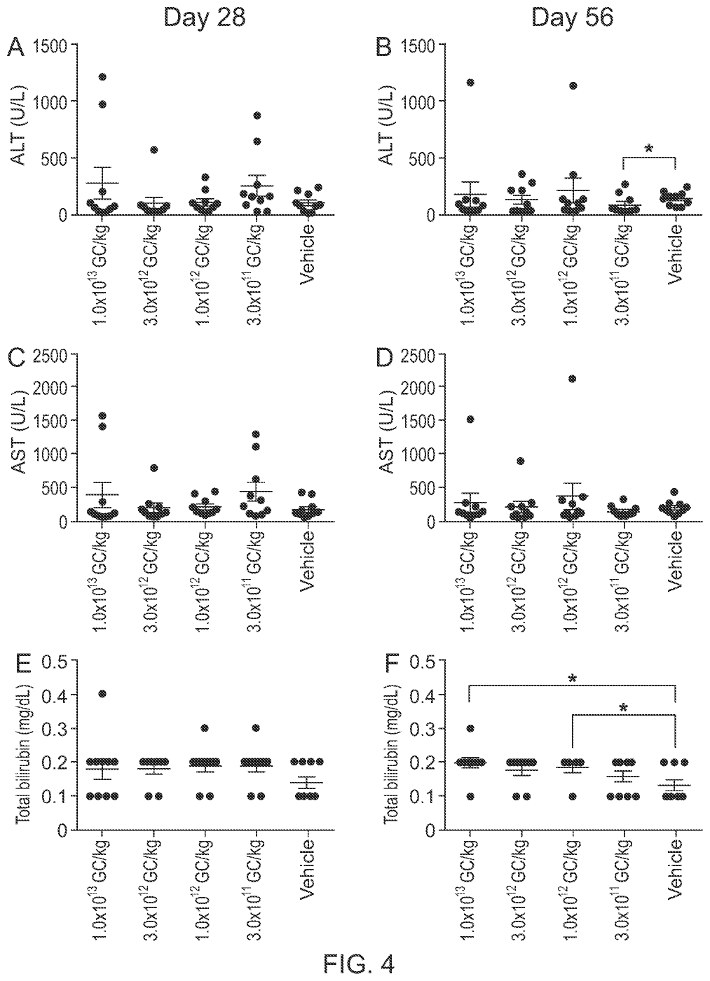

Serum chemistry panels were performed on samples collected at necropsy by Antech GLP (FIG. 4). Blood chemistry results were evaluated for statistical differences (p<0.05) in mice administered vector compared to vehicle controls on days 28 and 56 post-vector administration. To compare ALT and AST levels in vector- and vehicle control-administered mice, the Wilcoxon rank-sum test was applied. For mice necropsied at day 28, no significant differences in ALT or AST levels in mice administered any vector dose compared to those administered with the vehicle control was observed (FIGS. 4A, 4C). For mice necropsied at day 56, a significant reduction in ALT levels following administration of 3×10¹¹ GC/kg of the vector compared to the vehicle-treated cohort was observed (FIG. 4B) but no significant differences in AST levels were observed (FIG. 4D).

Serum total bilirubin levels were compared following administration of vector or vehicle control. Mice necropsied on day 28 displayed no significant differences (FIG. 4E). Mice necropsied at day 56 that were administered 1×10¹² GC/kg or 1×10¹³ GC/kg of vector exhibited a significant elevation in total bilirubin levels compared to the vehicle control-administered group (FIG. 4F).

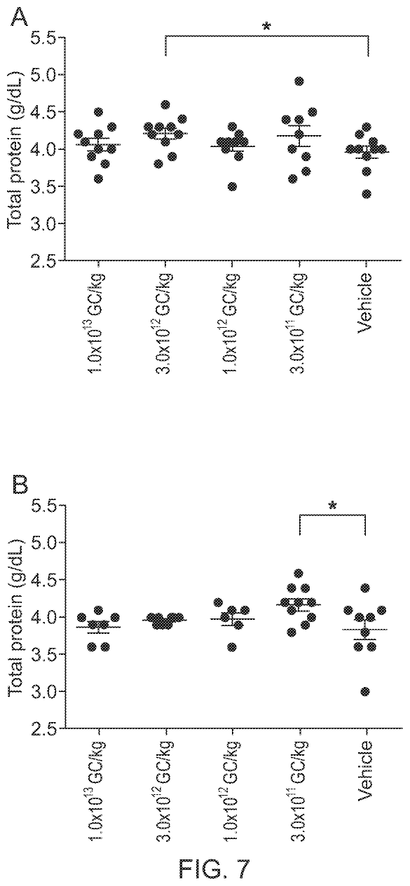

Additionally, comparing serum total protein levels revealed significant differences in mice administered with 3×10¹² GC/kg on day 28 (FIG. 7A) or 3×10¹¹ GC/kg of vector at day 56 (FIG. 7B).

Histopathological Findings

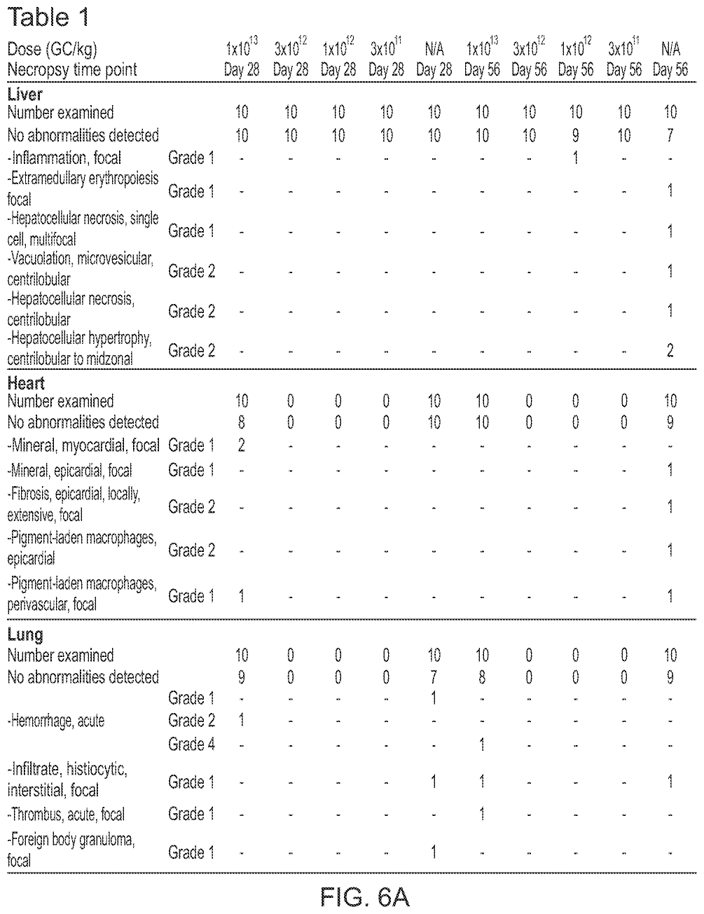

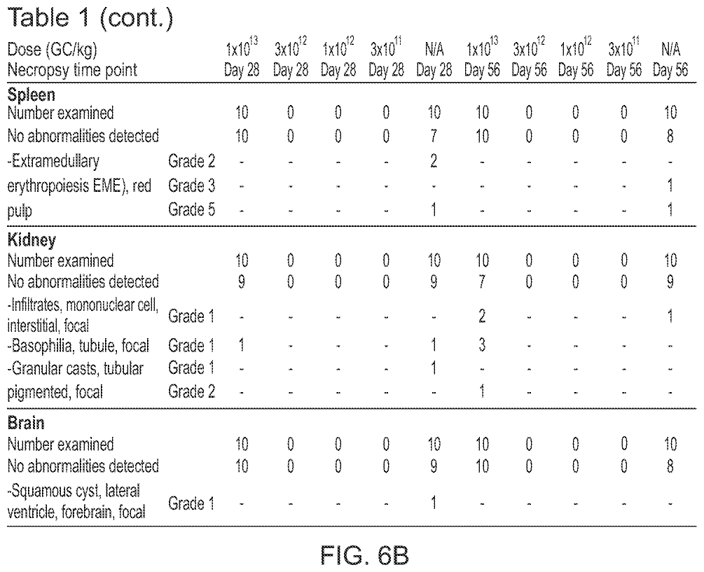

Tissues were harvested from all animals at the time of necropsy, stained with H&E, and a full histopathological analysis was performed. An experienced board-certified veterinary pathologist evaluated the liver sections in a blinded manner using predetermined scoring criteria. Histopathology slides for other tissues were evaluated and peer-reviewed for the highest vector dose group and the vehicle control group. No vector-related microscopic findings were observed (FIG. 6).

Most of the microscopic findings were observed in vehicle-administered animals and were considered potentially secondary to blood loss. However, no macroscopic or microscopic evidence of hemorrhage was observed (FIG. 6). These findings included centrilobular hepatocellular necrosis (ischemia), extramedullary erythropoiesis in the spleen and liver, epicardial fibrosis with pigment-laden macrophages (consistent with hemosiderin), pigment-laden macrophages with perivascular distribution in the heart, and pigmented (hemoglobin, presumptive) granular casts within renal tubules. Acute alveolar hemorrhage occurred in the lungs of some mice (1/10 mice administered with 1×10¹³ GC/kg necropsied at both day 28 and 56, 1/10 mice administered with vehicle control necropsied at day 28). With no histologic evidence of chronicity (e.g., hemosiderophages), we suspected that this finding was perimortem alveolar hemorrhage (potentially secondary to cardiac puncture). The observation of renal interstitial mononuclear cell infiltrates associated with minimal tubule basophilia was considered incidental. Other microscopic findings were incidental, background, or secondary to IV administration, and included myocardial/epicardial mineralization, pulmonary interstitial infiltrates, focal pulmonary foreign body granuloma (hair shaft), focal inflammation in the liver, mononuclear cell infiltrates within the kidney, and a squamous cyst in the brain. The mineralization observed in the heart of a vehicle-administered mouse and two mice from the high-dose group may represent mineralized thrombi, especially given the proximity of the lesions to blood vessels. We found a single acute non-occlusive fibrin thrombus in the lung of one mouse from the high-dose group. The hematopoietic infiltrates in the liver in one vehicle-administered animal were associated with minimal single hepatocellular necrosis.

Liver Vector GC and Transgene RNA Analysis

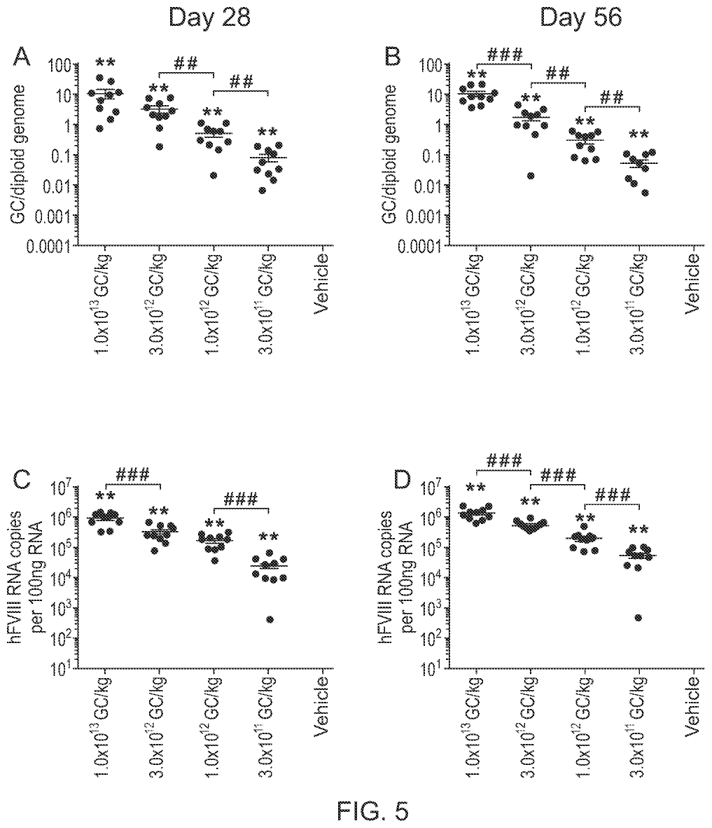

At the time of necropsy, liver was collected for biodistribution analysis. A dose-dependent increase was detected in both vector GC and hFVIII RNA levels in the liver (FIG. 5). Using a two-sample Wilcoxon rank-sum test, each vector-administered group was compared for mice necropsied on day 28 or 56 (FIGS. 5A, 5B). Significant differences were observed between doses in vector GCs for all vector-administered groups, except for mice administered 1×10¹³ GC/kg and 3×10¹² GC/kg and necropsied at day 28 (FIG. 5A).

The same comparisons for hFVIII RNA transcript levels between each vector-administered group were performed (two-sample Wilcoxon rank-sum test, FIG. 5C, 5D). For mice necropsied on day 28, all vector-administered groups displayed significant differences, except for the comparison between mice administered 3×10¹² GC/kg and 1×10¹² GC/kg (FIG. 5C). For mice necropsied on day 56, all vector-administered groups displayed significant differences in hFVIII RNA transcript levels (FIG. 5D).

Discussion

In this study, the MED of the clinical candidate vector, AAVhu37.E03.TTR.hFVIIIco-SQ.PA75, was determined in a hemophilia A mouse model. The experiments were chosen to be conducted in FVIII KO mice (rather than in wild-type C57BL/6 mice) for two reasons. First, using this strain of mice enabled the evaluation of efficacy in parallel with additional safety measurements. Second, the evaluation of potential vector-associated safety signs in the setting of any pathology associated with the defect in FVIII and the associated severe hemophilia and its sequelae could be determined. While it was not expected that the model would exhibit liver pathology, we were concerned that coagulation deficiencies could influence the response of the host liver to vector.

Following IV administration of AAVhu37.E03.TTR.hFVIIIco-SQ.PA75, hFVIII activity increased over the duration of the study from days 7 to 56, unless anti-hFVIII IgG antibodies developed in individual mice. Activity levels of >1.4 IU/ml (equivalent to 140% of normal) were detected at the time of necropsy. It is well established that FVIII levels directly correlate with clinical efficacy (Stonebraker J S et al., “A study of variations in the reported haemophilia A prevalence around the world,” Haemophilia 2010; 16:20-32). Indeed, hemophilia A patients are classified into different severity levels depending on the percentage of normal hFVIII; mild (5-40% of normal, 0.05-0.40 IU/ml), moderate (1-5% of normal, 0.01-0.05 IU/ml), and severe (>1%, 0.01 IU/ml). Given this, the results strongly suggest that the gene therapy product of the current disclosure would demonstrate clinical efficacy in hemophilia A patients.

While some FVIII KO mice did develop anti-hFVIII IgG antibodies, the relevance of this to the clinical application of this gene therapy approach is unknown as this vector expressed a human protein in mouse. Also, the region of the hFVIII protein to which the antibodies bound was not determined. Other investigators have used immune deficient FVIII KO mouse models (either Rag2−/− or CD4−/−) to evaluate expression and activity in the absence of antibody generation (Sabatino D E et al., “Animal models of hemophilia,” Prog Mol Biol Transl Sci 2012; 105:151-209; Bunting S et al., Mol Ther 2018; supra; Sabatino D E et al., “Efficacy and safety of long-term prophylaxis in severe hemophilia A dogs following liver gene therapy using AAV vectors,” Mol Ther 2011; 19:442-449; Riley B E et al., Blood 2016; supra).

No significant differences in liver transaminases were found in mice administered any vector dose compared to mice administered the vehicle control (except for one group administered 3×10¹¹ GC/kg). There was a significant elevation in total bilirubin levels following administration of 1×10¹² GC/kg or 1×10¹³ GC/kg of vector at day 56 compared to the vehicle-treated group.

Importantly, no gross or histological vector-related pathology findings were observed. The majority of the microscopic findings were in mice administered with the vehicle control and were considered to point to evidence of blood loss in conjunction with the clinical signs, although no direct evidence of hemorrhage or blood loss was observed.

As no dose-limiting vector-related safety measurements were observed, the maximally tolerated dose was greater than or equal to the highest dose tested, which was 1×10¹³ GC/kg. Moreover, conducting this study in a hemophilia A animal model allowed us to estimate the MED. Human FVIII activity was detected at all doses of the test article administered; hFVIII activity levels were significantly elevated for test article cohorts administered with greater than 3×10¹¹ GC/kg at all time points. Therefore, the MED is equal to 3×10¹¹ GC/kg.

At a higher dose of the factor VIII gene therapy vector BMN 270 (2×10¹³ vg/kg) 23.5% of normal hFVIII activity was achieved in DKO mice, which is substantially lower than levels achieved in the present experiments with the vector and murine model discussed herein at 1×10¹³ GC/kg (>140% of normal).

Due to the low MED for this treatment approach for hemophilia A, combined with expression data following systemic administration of the same vector in nonhuman primates (Greig J A et al., Hum Gene Ther 2018, supra), it is believed that this AAVhu37-based gene therapy approach has therapeutic potential in humans. In macaques, the hFVIII activity levels following IV administration of 1.2×10¹³ GC/kg would be sufficient to convert a severe hemophilia A phenotype (Greig J A et al., Hum Gene Ther 2018, supra), thereby reducing or potentially removing the need for recombinant hFVIII infusions.

### Example 2: Formulation of Solution for Intravenous (I.V.) Injection or Infusion

BAY 2599023 (AAVhu37.hFVIIIco) gene therapy AAV vector is formulated to a concentration of ≥5.0×10¹² genome copies (GC) BAY 2599023/mL in a solution of 20 mM Tris, 1 mM magnesium chloride (MgCl₂).6 H₂O, 200 mM sodium chloride (NaCl), containing 0.01% (Weight to volume) Pluronic® F-68 poloxamer, pH 8.0+/−0.2. The BAY 2599023 formulation is stored as a frozen liquid in a 2 mL 13 mm Type I clear glass vial at ≤−60° C. It is administered with a diluent, if necessary to obtain the desired therapeutic dose.

In another example, an AAV gene therapy vector, such as one useful for treatment of HA or SMA, is formulated to a concentration of 2.0×10¹³ vg/mL in 20 mM Tris pH 8.0, 1 mM MgCl₂, 200 mM NaCl, and 0.005% poloxamer 188.

### Example 3: Human Dosing Trials

BAY 2599023 (AAVhu37.hFVIIIco) is an adeno-associated virus (AAV) gene therapy vector, based on the AAVhu37 serotype. BAY 2599023 is a non-replicating AAV vector and contains a single-stranded DNA genome encoding a B-domain-deleted FVIII, under the control of a liver-specific promoter/enhancer combination, optimized for transgenic expression. The AAVhu37 capsid is a member of the hepatotropic clade E family. In preclinical studies it demonstrated efficient, liver-directed FVIII gene transfer, favorable biodistribution and durable FVIII expression.

Methods

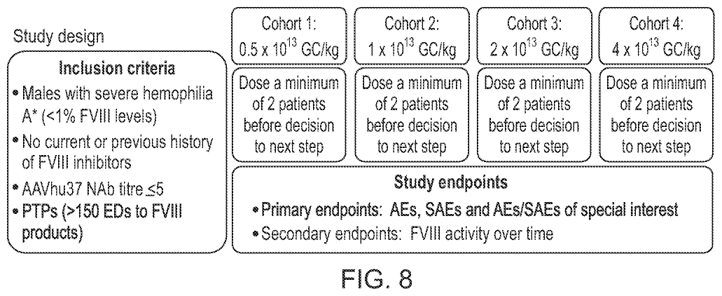

The BAY 2599023 phase ½, open-label, dose-finding study (identified in www.clinicaltrials.gov as trial no. NCT03588299) includes male patients aged ≥18 years with severe hemophilia A, no history of FVIII inhibitors, no detectable neutralizing immunity against AAVhu37 (neutralizing antibody titer ≤5), and ≥150 exposure days to FVIII products. Patients received a single intravenous infusion of BAY 2599023 and were enrolled sequentially into three dose cohorts (0.5×10¹³ GC/kg, 1.0×10¹³ GC/kg and 2.0×10¹³ GC/kg), each comprising at least two patients. Patients to be enrolled in a fourth cohort will receive a single infusion of 4×10¹³ GC/kg (See FIG. 8, study design). Primary endpoints were adverse events (AEs), serious AEs (SAEs) and AEs/SAEs of special interest (S/AESIs). The secondary endpoint was FVIII activity over time.

Results

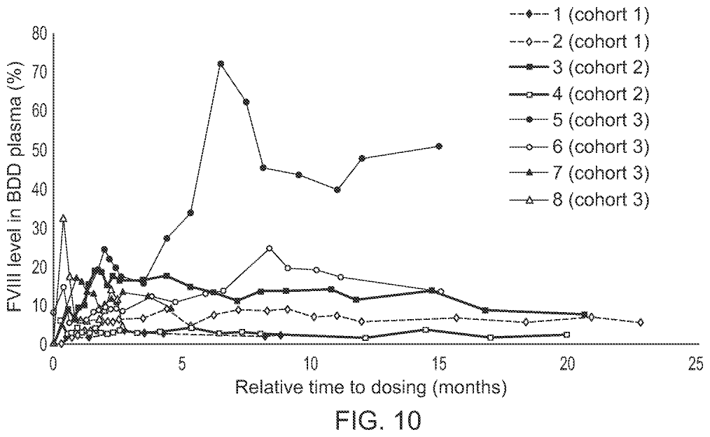

Three cohorts of ≥2 patients each (N=9) were enrolled sequentially (FIG. 8). Cohorts 1 and 2 each enrolled 2 patients; cohort 3 enrolled 5 patients. FIG. 10 presents FVIII activity data for the first eight patients, which shows that BAY 2599023 delivered sustained FVIII expression levels for up to >23 months, with evidence of bleed protection. Patients in Cohorts 2 and 3 have all been off prophylaxis with FVIII products since approximately 6-12 weeks after gene transfer. As of the treatment of patients 1-9, it has been observed that no spontaneous bleeds requiring treatment have been reported once FVIII levels >11 IU/dL were achieved. Of the 9 patients treated, 5 patients developed an AESI: Mild/moderate alanine aminotransferase (ALT) elevations observed in Cohort 2 (n=1) and Cohort 3 (n=3) were managed with corticosteroid treatment; another ALT elevation was reported as study-drug-related SAE in Cohort 3 (n=1) but returned to normal a few weeks after interruption of the H2 blocker famotidine.

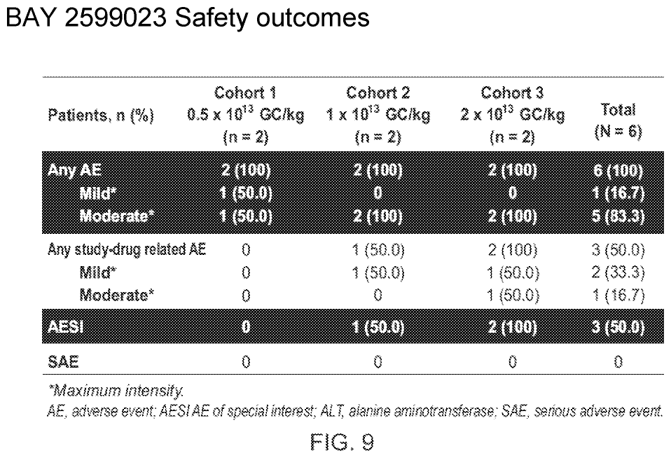

Detailed safety data was assembled after enrollment of the first 6 patients and is shown in FIG. 9. BAY 2599023 has a favorable safety profile. At the cut-off date (11 Jan. 2021), in Cohort 1, a follow up of 23 months of safety observation was reported with no SAEs, study-drug-related AEs or S/AESIs (FIG. 9). One patient dropped out for personal reasons after 12 months from treatment. In Cohort 2 (patient 3), a follow-up of 17 months of safety observation reported one AESI, mild elevation in ALT with no associated clinical symptoms or loss of FVIII activity levels. A short course of corticosteroid treatment resulted in a rapid return of ALT to the normal range. In Cohort 3, both patients had mild or moderate increases in transaminases without associated symptoms or loss of FVIII expression with ongoing corticosteroid treatment.

### CONCLUSIONS

In human dosing trials of BAY 2599023, all patients with evaluable data have shown effective, sustained FVIII levels, with asymptomatic ALT elevations that responded to corticosteroids. These results are evidence that BAY 2599023 is a key candidate in the evolution of gene therapy in hemophilia A.

### Exemplary Embodiments

Exemplary embodiments provided in accordance with the presently disclosed subject matter include, but are not limited to, the claims and the following embodiments:

1. A method for treating hemophilia A comprising administering to a patient in need thereof a dose, preferably a minimally effective dose, of an AAV gene therapy vector that delivers a human FVIII gene to a patient in need thereof, wherein the dose, preferably the minimally effective dose, is 3×10¹¹ genome copies/kg,

and optionally wherein the dose, preferably the minimally effective dose, when measured 56 days after dosage provides at least about 20% of normal human FVIII activity.

2. The method of embodiment 1, wherein the patient is converted from having severe hemophilia A to mild or moderate hemophilia A.

3. The method of any one of embodiments 1-2, wherein 60 days after administration the patient has 1% or more of normal human FVIII procoagulant activity.

4. The method of any one of embodiments 1-3, wherein the AAV gene therapy vector comprises an AAV capsid and a vector genome packaged therein, the vector genome comprising:

a. an AAV 5′-inverted terminal repeat (ITR) sequence;

b. a liver-specific promoter

c. a coding sequence encoding a human FVIII having FVIII procoagulant function; and

d. an AAV 3″-ITR sequence, wherein the coding sequence preferably comprises the nucleic acid sequence that is at least 90, 91, 92, 93, 94, 95, 96, 97, 98, 99, or 100% identical to SEQ ID NO: 2.

5. The method of any one of embodiments 1-4, wherein the AAV gene therapy vector comprises an AAV capsid is a hu.37 capsid.

6. The method of any one of embodiments 1-5, wherein the AAV gene therapy vector comprises an AAV 5′-ITR from AAV2.

7. The method of any one of embodiments 1-6, wherein the AAV gene therapy vector comprises an AAV 5′-ITR that comprises the nucleotide sequence as set forth in SEQ ID NO: 11.

8. The method of any one of embodiments 1-7, wherein the AAV gene therapy vector comprises an AAV 3 ‘-ITR from AAV2.

9. The method of embodiment 8, wherein the AAV 3’-ITR comprises the nucleotide sequence as set forth in SEQ ID NO: 12.

10. The method of any one of embodiments 1-9, wherein the AAV gene therapy vector comprises AAV 5′-ITR and AAV 3′-ITR from AAV2.

11. The method of any one of embodiments 1-10, wherein the AAV gene therapy vector comprises a liver-specific promoter that is a transthyretin (TTR) promoter.

12. The method of embodiment 11, wherein the TTR promoter comprises the nucleotide sequence as set forth in SEQ ID NO: 7.

13. The method of any one of embodiments 1-12, wherein the AAV gene therapy vector comprises a vector genome that comprises an enhancer.

14. The method of embodiment 13, wherein the enhancer is a liver-specific enhancer.

15. The method of embodiment 14, wherein the liver-specific enhancer is a transthyretin enhancer (enTTR).

16. The method of embodiment 15, wherein the enTTR comprises the nucleotide sequence as set forth in SEQ ID NO: 5.

17. The method of any one of embodiments 1-16, wherein the AAV gene therapy vector comprises a vector genome that comprises a polyA sequence.

18. The method of embodiment 18, wherein the polyA sequence comprises the nucleotide sequence as set forth in SEQ ID NO: 10.

19. The method of any one of embodiments 1-18, wherein the AAV gene therapy vector has a viral genome that is 5 kilobases to 5.5 kilobases in size.

20. The method of any one of embodiments 1-19, wherein the AAV gene therapy vector comprises AAVhu37.E03.TTR.hFVIIIco-SQ.PA75 (SEQ ID NO: 13).

21. The method of any one of embodiments 1-20, wherein the AAV gene therapy vector is BAY 2599023.

22. The method of any one of embodiments 1-3, wherein the AAV gene therapy vector is BAY 2599023, SPK-8011, Valoctocogene roxaparvovec, or Giroctocogene fitelparvovec.

23. A method for administering a therapeutically effective dose of an AAV gene therapy vector that delivers a human FVIII gene for treatment of hemophilia A comprising obtaining measurements of FVIII activity in a study of at least 100 male mice having a disease pathology for bleeding and who have received injections of the AAV gene therapy vector and obtaining the minimally effective dose calculated from those measurements; and

administering to a patient in need thereof the minimally effective dose of the AAV gene therapy vector,

optionally wherein the minimally effective dose when measured 56 days after dosage provides at least about 20% of normal human FVIII activity.

24. The method of embodiment 23, wherein the male mice having a disease pathology for bleeding are factor VIII knock out mice.

25. The method of any one of embodiments 23-24, wherein the method shows no liver toxicity effects.

26. The method of any one of embodiments 23-25, wherein the patient is converted from having severe hemophilia A to mild or moderate hemophilia A.

27. The method of any one of embodiments 23-24, wherein 60 days after administration the patient has 1% or more of normal human FVIII procoagulant activity.

28. The method of any one of embodiments 23-25, wherein the AAV gene therapy vector comprises an AAV capsid and a vector genome packaged therein, the vector genome comprising:

a. an AAV 5′-inverted terminal repeat (ITR) sequence;

b. a liver-specific promoter

c. a coding sequence encoding a human FVIII having FVIII procoagulant function; and

d. an AAV 3″-ITR sequence,

wherein the coding sequence preferably comprises the nucleic acid sequence that is at least 90, 91, 92, 93, 94, 95, 96, 97, 98, 99, or 100% identical to SEQ ID NO: 2.

29. The method of any one of embodiments 23-28, wherein the AAV gene therapy vector comprises an AAV capsid is a hu.37 capsid.

30. The method of any one of embodiments 23-29, wherein the AAV gene therapy vector comprises an AAV 5′-ITR from AAV2.

31. The method of any one of embodiments 23-30, wherein the AAV gene therapy vector comprises an AAV 5′-ITR that comprises the nucleotide sequence as set forth in SEQ ID NO: 11.

32. The method of any one of embodiments 23-31, wherein the AAV gene therapy vector comprises an AAV 3′-ITR from AAV2.

33. The method of embodiment 32, wherein the AAV 3′-ITR comprises the nucleotide sequence as set forth in SEQ ID NO: 12.

34. The method of any one of embodiments 23-33, wherein the AAV gene therapy vector comprises AAV 5′-ITR and AAV 3′-ITR from AAV2.

35. The method of any one of embodiments 23-34, wherein the AAV gene therapy vector comprises a liver-specific promoter that is a transthyretin (TTR) promoter.

36. The method of embodiment 35, wherein the TTR promoter comprises the nucleotide sequence as set forth in SEQ ID NO: 7.

37. The method of any one of embodiments 23-36, wherein the AAV gene therapy vector comprises a vector genome that comprises an enhancer.

38. The method of embodiment 37, wherein the enhancer is a liver-specific enhancer.

39. The method of embodiment 38, wherein the liver-specific enhancer is a transthyretin enhancer (enTTR).

40. The method of embodiment 39, wherein the enTTR comprises the nucleotide sequence as set forth in SEQ ID NO: 5.

41. The method of any one of embodiment 23-40, wherein the AAV gene therapy vector comprises a vector genome that comprises a polyA sequence.

42. The method of embodiment 41, wherein the polyA sequence comprises the nucleotide sequence as set forth in SEQ ID NO: 10.

43. The method of any one of embodiments 23-42, wherein the AAV gene therapy vector has a viral genome that is 5 kilobases to 5.5 kilobases in size.

44. The method of any one of embodiments 23-43, wherein the AAV gene therapy vector comprises AAVhu37.E03.TTR.hFVIIIco-SQ.PA75 (SEQ ID NO: 13).

45. The method of any one of embodiments 23-44, wherein the AAV gene therapy vector is BAY 2599023.

46. The method of any one of embodiments 23-25, wherein the AAV gene therapy vector is BAY 2599023, SPK-8011, Valoctocogene roxaparvovec, or Giroctocogene fitelparvovec.

47. The method of any one of embodiments 23-46, wherein the method results in alanine aminotransferase (ALT) significantly reduced compared to a vehicle-treated control

48. The method of any one of embodiments 23-47, wherein the method results in no significant elevation in total bilirubin levels 56 days after administration compared to a vehicle-treated control.

49. The method of any one of embodiments 23-48, wherein the minimally effective dosage is less than 4.0×10¹² genome copies/kg.

50. The method of embodiment 49, wherein 19 months after administration sustained human FVIII activity levels are achieved.

51. The method of embodiment 50, wherein the sustained human FVIII activity levels are at least 1% or at least 5% of normal human FVIII activity levels, preferably between 5 and 10% of normal human FVIII activity levels.

52. The method of any one of embodiments 23-51, wherein the dose is selected from the group consisting of 1.5×10¹², 1.6×10¹², 1.7×10¹², 1.8×10¹², 1.9×10¹², 2.0×10¹², 2.1×10¹², 2.2×10¹², 2.3×10¹², 2.4×10¹², 2.5×10¹², 2.6×10¹², 2.7×10¹², 2.8×10¹², 2.9×10¹², 3.0×10¹², 3.1×10¹², 3.2×10¹², 3.3×10¹², 3.4×10¹², 3.5×10¹², 3.6×10¹², 3.7×10¹², 3.8×10¹², and 3.9×10¹² genome copies/kg.

53. A method for determining a minimally effective dosage of an AAV gene therapy vector for delivering human FVIII; the method comprising:

obtaining at least 50 male knock out FVIII mice;

injecting the male knock out FVIII mice with an IV tail vein injection of either (a) the AAV gene therapy vector at one of four doses: 3×10¹¹, 1×10¹², 3×10¹², or 1×10¹³ GC/kg or (b) a vehicle control; wherein the mice receiving the AAV gene therapy vector are divided into four cohorts and each cohort receives a different one of the four doses;

performing a first and a second necropsy; wherein the first necropsy occurs on a first group of mice on a day between 23-33 and the second necropsy occurs on a second group of mice on a day between 51-61 after injection;

measuring hFVIII activity with each necropsy;

determining peak and long term hFVIII activity from the hFVIII activity measurements; and

calculating minimally effective dosage from the peak and long term hFVIII activity.

54. The method of embodiment 53, wherein the second necropsy shows average peak expression levels in the high-dose group (1×10¹³ GC/kg) of 1.4 IU/ml at the first necropsy and 1.4 IU/ml at the second necropsy.

55. The method of any one of embodiments 53-54, wherein in the first necropsy no significant difference in ALT or AST level is present for mice receiving an AAV vector dose.

56. The method of any one of embodiments 53-55, wherein the AAV gene therapy vector is AAVhu37.E03.TTR.hFVIIIco-SQ.PA75 (SEQ ID NO:13), Valoctocogene roxaparvovec, SPK-8011 or Giroctocogene fitelparvovec, preferably AAVhu37.E03.TTR.hFVIIIco-SQ.PA75 (SEQ ID NO:13).

57. The method of any one of embodiments 53-56, wherein the AAV gene therapy vector comprises an AAVhu37 capsid encoding an amino acid sequence having 90%, 95%, or 99% homology to the amino acid sequence of SEQ ID NO: 22, a transgene encoding an active human FVIII polypeptide, and additional vector control sequences.

58. A method of treatment for hemophilia A comprising administering a therapeutically effective dose of an AAV vector that delivers a human FVIII gene to a subject in need thereof, wherein said AAV vector is administered as a stable liquid pharmaceutical formulation, and wherein the stable liquid pharmaceutical formulation of the AAV vector comprises:
(a) the AAV vector at a concentration from about 1×10¹² vg/ml to 1×10¹³ vg/ml;
(b) from about 10 mM to 30 mM Tris;
(c) from about 150 mM to 300 mM NaCl;
(d) from about 0.5 mM to 3.0 mM MgCl₂.6H₂O; and
(e) from about 0.002% (w/v) to 0.02% (w/v) poloxamer 188, such as Pluronic® F-68;

wherein the pharmaceutical formulation has a pH of from 7.8 to 8.2.

59. The method of embodiment 58, wherein the AAV vector is AAV serotype hu37 comprising:
i) an AAV 5′-inverted terminal repeat (ITR) sequence;
ii) a liver-specific promoter;
iii) a liver specific enhancer;
iv) a coding sequence encoding a human FVIII having FVIII procoagulant function; and
v) an AAV 3″-ITR sequence.

60. The method of embodiments 58 or 59, wherein the AAV vector is AAV serotype hu37 comprising:

i) an AAV 5′-inverted terminal repeat (ITR) sequence;

ii) a liver-specific promoter;

iii) a liver-specific enhancer comprising a transthyretin enhancer (enTTR);

iv) a coding sequence encoding a human FVIII having FVIII procoagulant function; and
v) an AAV 3″-ITR sequence, wherein the coding sequence preferably comprises the nucleic acid sequence that is at least 90, 91, 92, 93, 94, 95, 96, 97, 98, 99, or 100% identical to SEQ ID NO: 2.

61. The method of any of embodiments 58 to 60, wherein the AAV vector is AAV serotype hu37 comprising
i) an AAV 5′-inverted terminal repeat (ITR) sequence comprising the AAV2 5′ITR as set forth in SEQ ID NO: 11;
ii) a liver-specific promoter, comprising the TTR promoter as set forth in SEQ ID NO: 7;
iii) a liver-specific enhancer as set forth in SEQ ID NO: 5;
iv) a coding sequence encoding a human FVIII having FVIII procoagulant function; and
   an AAV 3″-ITR sequence, wherein the coding sequence preferably comprises the nucleic acid sequence that is at least 90, 91, 92, 93, 94, 95, 96, 97, 98, 99, or 100% identical to SEQ ID NO: 2.

62. The method of any of embodiments 58 to 61, wherein the AAV vector comprises wherein the AAV vector comprises AAVhu37.E03.TTR.hFVIIIco-SQ.PA75 (SEQ ID NO: 13).

63. The method of any of embodiments 58 to 61 wherein the stable liquid pharmaceutical formulation of the AAV vector comprises:
(a) the AAV vector at a concentration of from 5.0×10¹² to 1×10¹³ GC/ml;
(b) about 20 mM Tris;
(c) about 200 mM NaCl;
(d) about 1.0 mM MgCl₂.6H₂O; and
(e) about 0.01% (w/v) poloxamer 188, such as Pluronic® F-68;

wherein the pharmaceutical formulation has a pH of about 8.0.

64. The method of any of embodiments 58 to 63 wherein the stable liquid pharmaceutical formulation comprises the AAV vector of AAV serotype hu37 and the pH is between 7.5 and 8.0.

1. A method for treating hemophilia A comprising administering to a patient in need thereof a dose of from 0.5×10¹³ to 4×10¹³ genome copies/kg of an AAV gene therapy vector for delivering human FVIII or a variant thereof;
wherein sustained human FVIII procoagulant activity is achieved as measured 10 months after administration and optionally wherein the dosage provides clinically proven effectiveness.

2. The method of claim 1, wherein the sustained human FVIII levels are at least 1% of normal human FVIII levels, preferably between 1 and 5% or greater than or equal to 5% of normal human FVIII levels.

3. The method of claim 1, wherein the patient is converted from having severe hemophilia A to mild or moderate hemophilia A.

4. The method claim 1, wherein 60 days after administration the patient has 1% or more of normal human FVIII procoagulant activity.

5. The method of claim 1, wherein the AAV gene therapy vector comprises an AAV capsid and a vector genome packaged therein, the vector genome comprising:
a. an AAV 5′-inverted terminal repeat (ITR) sequence;
b. a liver-specific promoter
c. a coding sequence encoding a human FVIII having FVIII procoagulant function; and
d. an AAV 3″-ITR sequence,
wherein the coding sequence preferably comprises the nucleic acid sequence that is at least 90, 91, 92, 93, 94, 95, 96, 97, 98, 99, or 100% identical to SEQ ID NO: 2.

6. The method of claim 1, wherein the AAV gene therapy vector comprises an AAV capsid is a hu.37 capsid.

7. The method of claim 1, wherein the AAV gene therapy vector comprises an AAV 5′-ITR from AAV2.

8. The method of claim 1, wherein the AAV gene therapy vector comprises an AAV 5′-ITR that comprises the nucleotide sequence as set forth in SEQ ID NO: 11.

9. The method of claim 1, wherein the AAV gene therapy vector comprises an AAV 3′-ITR from AAV2.

10. The method of claim 9, wherein the AAV 3′-ITR comprises the nucleotide sequence as set forth in SEQ ID NO: 12.

11. The method of claim 1, wherein the AAV gene therapy vector comprises AAV 5′-ITR and AAV 3′-ITR from AAV2.

12. The method of claim 1, wherein the AAV gene therapy vector comprises a liver-specific promoter that is a transthyretin (TTR) promoter.

13. The method of claim 12, wherein the TTR promoter comprises the nucleotide sequence as set forth in SEQ ID NO: 7.

14. The method of claim 1, wherein the AAV gene therapy vector comprises a vector genome that comprises an enhancer.

15. The method of claim 14, wherein the enhancer is a liver-specific enhancer.

16. The method of claim 15, wherein the liver-specific enhancer is a transthyretin enhancer (enTTR).

17. The method of claim 16, wherein the enTTR comprises the nucleotide sequence as set forth in SEQ ID NO: 5.

18. The method of claim 1, wherein the AAV gene therapy vector comprises a vector genome that comprises a polyA sequence.

19. The method of claim 18, wherein the polyA sequence comprises the nucleotide sequence as set forth in SEQ ID NO: 10.

20. The method of claim 1, wherein the AAV gene therapy vector has a viral genome that is 5 kilobases to 5.5 kilobases in size.

21. The method of claim 1, wherein the AAV gene therapy vector comprises AAVhu37.E03.TTR.hFVIIIco-SQ.PA75 (SEQ ID NO:13)

22. The method of claim 1, wherein the AAV gene therapy vector is BAY 2599023.

23. The method of claim 1, wherein the AAV gene therapy vector is BAY 2599023, Valoctocogene roxaparvovec, or Giroctocogene fitelparvovec.

24. The method of claim 1, wherein the dose is selected from the group consisting of 0.5×10¹³, 0.6×10¹³, 0.7×10¹³, 0.8×10¹³, 0.9×10¹³, 1.0×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2.0×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3.0×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, and 4.0×10¹³ genome copies/kg.

25. A method of treatment for hemophilia A comprising: administering a therapeutically effective dose of an AAV vector that delivers a human FVIII gene to a subject in need thereof, wherein said AAV vector is administered as a stable liquid pharmaceutical formulation, and wherein the stable liquid pharmaceutical formulation of the AAV vector comprises:
a. the AAV vector at a concentration from about 1×10¹² vg/ml to 1×10¹³ vg/ml;
b. from about 10 mM to 30 mM Tris;
c. from about 150 mM to 300 mM NaCl;
d. from about 0.5 mM to 3.0 mM MgCl₂.6H₂O; and
e. from about 0.002% (w/v) to 0.02% (w/v) poloxamer 188, such as Pluronic® F-68;
wherein the pharmaceutical formulation has a pH of from 7.8 to 8.2.

26. The method of claim 25, wherein the AAV vector is AAV serotype hu37 comprising:
a. an AAV 5′-inverted terminal repeat (ITR) sequence;
b. a liver-specific promoter;
c. a liver specific enhancer;
d. a coding sequence encoding a human FVIII having FVIII procoagulant function; and
e. an AAV 3″-ITR sequence

27. The method of claim 25, wherein the AAV vector is AAV serotype hu37 comprising:
a. an AAV 5′-inverted terminal repeat (ITR) sequence;
b. a liver-specific promoter;
c. a liver-specific enhancer comprising a transthyretin enhancer (enTTR);
d. a coding sequence encoding a human FVIII having FVIII procoagulant function; and
e. an AAV 3″-ITR sequence, wherein the coding sequence preferably comprises the nucleic acid sequence that is at least 90, 91, 92, 93, 94, 95, 96, 97, 98, 99, or 100% identical to SEQ ID NO: 2.

28. The method of claim 25, wherein the AAV vector is AAV serotype hu37 comprising:
a. an AAV 5′-inverted terminal repeat (ITR) sequence comprising the AAV2 5′ITR as set forth in SEQ ID NO: 11;
b. a liver-specific promoter, comprising the TTR promoter as set forth in SEQ ID NO: 7;
c. a liver-specific enhancer as set forth in SEQ ID NO: 5;
d. a coding sequence encoding a human FVIII having FVIII procoagulant function; and
e. an AAV 3″-ITR sequence, wherein the coding sequence preferably comprises the nucleic acid sequence that is at least 90, 91, 92, 93, 94, 95, 96, 97, 98, 99, or 100% identical to SEQ ID NO: 2.

29. The method of claim 25, wherein the AAV vector comprises wherein the AAV vector comprises AAVhu37.E03.TTR.hFVIIIco-SQ.PA75 (SEQ ID NO: 13).

30. The method of claim 25, wherein the stable liquid pharmaceutical formulation of the AAV vector comprises:
a. the AAV vector at a concentration of from 5.0×10¹² to 1×10¹³ vg/ml;
b. about 20 mM Tris;
c. about 200 mM NaCl;
d. about 1.0 mM MgCl₂.6H₂O; and
e. about 0.01% (w/v) poloxamer 188, such as Pluronic® F-68;
wherein the pharmaceutical formulation has a pH of about 8.0.

31. The method of claim 25, wherein the stable liquid pharmaceutical formulation comprises the AAV vector of AAV serotype hu37 and the pH is between 7.5 and 8.0.


SEQUENCE LISTING (22 sequences)

SEQ ID NO: 1 - DNA, 4371 bp, Homo sapiens
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 2 - DNA, 4374 bp, Artificial Sequence
  constructed sequence
atgcagatcgagctgagcacctgcttcttcctgtgcctgctgcggttctgcttctccgccacccggcggtactacctgggagccgtggagctgagctgggattacatgcagagcgatctgggagagctgccagtggatgcccggttcccaccacgggtgccaaagagcttcccattcaacaccagcgtggtgtacaagaagaccctgttcgtggagttcaccgatcacctgttcaacatcgccaagccacggccaccctggatgggactgctgggaccaaccatccaggccgaggtgtacgataccgtggtgatcaccctgaagaacatggcctctcatcctgtgtccctgcacgccgtgggagtgagctactggaaggccagcgagggagccgagtacgatgatcagaccagccagcgggagaaggaggatgataaggtgttcccaggaggaagccacacctacgtgtggcaggtgctgaaggagaacggaccaatggccagcgatccactgtgcctgacctacagctacctgagccacgtggatctggtgaaggatctgaacagcggactgatcggagccctgctggtgtgccgggagggaagcctggccaaggagaagacccagaccctgcacaagttcatcctgctgttcgccgtgttcgatgagggaaagagctggcacagcgagaccaagaacagcctgatgcaggatcgggatgccgccagcgcccgggcctggccaaagatgcacaccgtgaacggatacgtgaaccggagcctgccaggactgatcggatgccaccggaagagcgtgtactggcacgtgatcggaatgggaaccaccccagaggtgcactctatcttcctggagggacacacctttctggtgcggaaccaccggcaggccagcctggagatcagcccaatcaccttcctgaccgcccagaccctgctgatggatctgggacagttcctgctgttctgccatatcagcagccaccagcacgatggaatggaggcctacgtgaaggtggatagctgcccagaggagccacagctgcggatgaagaacaacgaggaggccgaggattacgatgatgatctgaccgatagcgagatggatgtggtgcggttcgatgatgataacagcccaagcttcatccagatccggagcgtggccaagaagcacccaaagacctgggtgcactacatcgccgccgaggaggaggattgggattacgccccactggtgctggcccctgatgatcggagctacaagagccagtacctgaacaacggaccacagcggatcggacggaagtacaaaaaagtgcggttcatggcctacaccgatgagaccttcaagacccgggaggccatccagcacgagagcggaatcctgggaccactgctgtacggagaggtgggagataccctgctgatcatcttcaagaaccaggccagccggccatacaacatctacccacacggaatcaccgatgtgcggccactgtacagccggcggctgccaaagggagtgaagcacctgaaggatttcccaatcctgccaggagagatcttcaagtacaagtggacagtgacagtggaggatggaccaaccaagtctgatccaagatgcctgaccagatactacagcagctttgtgaacatggagagagacctggcctctggactgattggaccactgctgatctgctacaaggagtctgtggatcagagaggaaaccagatcatgtctgataagagaaatgtgatcctgttctctgtgtttgatgagaacagaagctggtacctgacagagaacatccagagattcctgccaaacccagccggagtgcagctggaggatccagagttccaggccagcaacatcatgcacagcatcaacggatacgtgttcgatagcctgcagctgagcgtgtgcctgcacgaggtggcctattggtatatcctgagcatcggagcccagaccgatttcctgagcgtgttcttcagcggatacaccttcaagcacaagatggtgtacgaggataccctgaccctgttcccattctccggagagaccgtgttcatgagcatggagaacccaggactgtggatcctgggatgccacaactctgatttcagaaacagaggaatgactgccctgctgaaagtgtccagctgtgataagaacactggagattactatgaggatagctatgaggatatctctgcctacctgctgagcaagaacaatgccattgagccaagaagcttcagccagaacccaccagtgctgaagagacaccagagagagatcaccagaaccaccctgcagtctgatcaggaggagattgattatgatgataccatctctgtggagatgaagaaggaggattttgatatctatgatgaggatgagaaccagagcccaagaagcttccagaagaagaccagacactacttcatcgctgcagtggagagactgtgggattatggaatgagcagcagcccacacgtgctgagaaacagagcccagagcggatctgtgccacagttcaagaaggtggtgttccaggagttcaccgatggaagcttcacccagccactgtaccggggagagctgaacgagcacctgggactgctgggaccatacatccgggccgaggtggaggataacatcatggtgaccttccggaaccaggccagccggccatacagcttctacagcagcctgatcagctacgaggaggatcagcggcagggagccgagccacggaagaacttcgtgaagccaaacgagaccaagacctacttctggaaggtgcagcaccacatggccccaaccaaggatgagttcgattgcaaggcctgggcctacttcagcgatgtggatctggagaaggatgtgcacagcggactgatcggaccactgctggtgtgccacaccaacaccctgaacccagcccacggacggcaggtgaccgtgcaggagttcgccctgttcttcaccatcttcgatgagaccaagagctggtacttcaccgagaacatggagcggaactgccgggccccttgcaacatccagatggaggatccaaccttcaaggagaactaccggttccacgccatcaacggatacatcatggataccctgccaggactggtgatggcccaggatcagcggatccggtggtacctgctgagcatgggaagcaacgagaacatccacagcatccacttcagcggacacgtgttcaccgtgcggaagaaggaggagtacaagatggccctgtacaacctgtacccaggagtgttcgagaccgtggagatgctgccaagcaaggccggaatctggcgggtggagtgcctgatcggagagcacctgcacgccggaatgagcaccctgttcctggtgtacagcaacaagtgccagaccccactgggaatggccagcggacacatccgggatttccagatcaccgccagcggacagtacggacagtgggccccaaagctggcccggctgcactacagcggaagcatcaacgcctggagcaccaaggagccattcagctggatcaaagtggatctgctggccccaatgatcatccacggaatcaagacccagggagcccggcagaagttcagcagcctgtacatcagccagttcatcatcatgtacagcctggatggaaagaagtggcagacctaccggggaaacagcaccggaaccctgatggtgttcttcggaaacgtggatagcagcggaatcaagcacaacatcttcaacccaccaatcatcgcccgatacatccggctgcacccaacccactacagcatcagaagcaccctgcggatggagctgatgggatgtgatctgaacagctgctccatgccactgggaatggagagcaaggccatcagcgatgcccagatcaccgccagcagctacttcaccaacatgttcgccacctggagcccaagcaaggcccggctgcacctgcagggacggagcaacgcctggcggccacaggtgaataacccaaaggagtggctgcaggtggatttccagaagaccatgaaggtgaccggagtgaccacccagggagtgaagagcctgctgactagcatgtatgtgaaggagttcctgatcagcagcagccaggatggacaccagtggaccctgttcttccagaacggaaaggtgaaggtgttccagggaaaccaggatagcttcaccccagtggtgaacagcctggatccaccactgctgacccgatacctgcggatccacccacagagctgggtgcaccagatcgccctgagaatggaggtgctgggatgcgaggcccaggatctgtactga

SEQ ID NO: 3 - protein, 1457 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - DNA, 34 bp, Artificial Sequence
  constructed sequence
tgtttgctgcttgcaatgtttgcccattttaggg

SEQ ID NO: 5 - DNA, 100 bp, Artificial Sequence
  constructed sequence
ctacctcgtgatcgcccggcccctgttcaaacatgtcctaatactctgtctctgcaagggtcatcagtagttttccatcttactcaacatcctcccagtg

SEQ ID NO: 6 - DNA, 42 bp, Artificial Sequence
  constructed sequence
aggttaatttttaaactgtttgctctggttaataatctcagg

SEQ ID NO: 7 - DNA, 190 bp, Artificial Sequence
  constructed sequence
atttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 8 - DNA, 176 bp, Artificial Sequence
  constructed sequence
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 9 - DNA, 218 bp, Artificial Sequence
  constructed sequence
tggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaata

SEQ ID NO: 10 - DNA, 75 bp, Artificial Sequence
  constructed sequence
aataaagtctgagtgggcggcagcctgtgtgtgcctgggttctctctgtcccggaatgtgcaaacaatggaggtg

SEQ ID NO: 11 - DNA, 168 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctact

SEQ ID NO: 12 - DNA, 164 bp, Artificial Sequence
  constructed sequence
gataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 7920 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgcagatcgagctgagcacctgcttcttcctgtgcctgctgcggttctgcttctccgccacccggcggtactacctgggagccgtggagctgagctgggattacatgcagagcgatctgggagagctgccagtggatgcccggttcccaccacgggtgccaaagagcttcccattcaacaccagcgtggtgtacaagaagaccctgttcgtggagttcaccgatcacctgttcaacatcgccaagccacggccaccctggatgggactgctgggaccaaccatccaggccgaggtgtacgataccgtggtgatcaccctgaagaacatggcctctcatcctgtgtccctgcacgccgtgggagtgagctactggaaggccagcgagggagccgagtacgatgatcagaccagccagcgggagaaggaggatgataaggtgttcccaggaggaagccacacctacgtgtggcaggtgctgaaggagaacggaccaatggccagcgatccactgtgcctgacctacagctacctgagccacgtggatctggtgaaggatctgaacagcggactgatcggagccctgctggtgtgccgggagggaagcctggccaaggagaagacccagaccctgcacaagttcatcctgctgttcgccgtgttcgatgagggaaagagctggcacagcgagaccaagaacagcctgatgcaggatcgggatgccgccagcgcccgggcctggccaaagatgcacaccgtgaacggatacgtgaaccggagcctgccaggactgatcggatgccaccggaagagcgtgtactggcacgtgatcggaatgggaaccaccccagaggtgcactctatcttcctggagggacacacctttctggtgcggaaccaccggcaggccagcctggagatcagcccaatcaccttcctgaccgcccagaccctgctgatggatctgggacagttcctgctgttctgccatatcagcagccaccagcacgatggaatggaggcctacgtgaaggtggatagctgcccagaggagccacagctgcggatgaagaacaacgaggaggccgaggattacgatgatgatctgaccgatagcgagatggatgtggtgcggttcgatgatgataacagcccaagcttcatccagatccggagcgtggccaagaagcacccaaagacctgggtgcactacatcgccgccgaggaggaggattgggattacgccccactggtgctggcccctgatgatcggagctacaagagccagtacctgaacaacggaccacagcggatcggacggaagtacaaaaaagtgcggttcatggcctacaccgatgagaccttcaagacccgggaggccatccagcacgagagcggaatcctgggaccactgctgtacggagaggtgggagataccctgctgatcatcttcaagaaccaggccagccggccatacaacatctacccacacggaatcaccgatgtgcggccactgtacagccggcggctgccaaagggagtgaagcacctgaaggatttcccaatcctgccaggagagatcttcaagtacaagtggacagtgacagtggaggatggaccaaccaagtctgatccaagatgcctgaccagatactacagcagctttgtgaacatggagagagacctggcctctggactgattggaccactgctgatctgctacaaggagtctgtggatcagagaggaaaccagatcatgtctgataagagaaatgtgatcctgttctctgtgtttgatgagaacagaagctggtacctgacagagaacatccagagattcctgccaaacccagccggagtgcagctggaggatccagagttccaggccagcaacatcatgcacagcatcaacggatacgtgttcgatagcctgcagctgagcgtgtgcctgcacgaggtggcctattggtatatcctgagcatcggagcccagaccgatttcctgagcgtgttcttcagcggatacaccttcaagcacaagatggtgtacgaggataccctgaccctgttcccattctccggagagaccgtgttcatgagcatggagaacccaggactgtggatcctgggatgccacaactctgatttcagaaacagaggaatgactgccctgctgaaagtgtccagctgtgataagaacactggagattactatgaggatagctatgaggatatctctgcctacctgctgagcaagaacaatgccattgagccaagaagcttcagccagaacccaccagtgctgaagagacaccagagagagatcaccagaaccaccctgcagtctgatcaggaggagattgattatgatgataccatctctgtggagatgaagaaggaggattttgatatctatgatgaggatgagaaccagagcccaagaagcttccagaagaagaccagacactacttcatcgctgcagtggagagactgtgggattatggaatgagcagcagcccacacgtgctgagaaacagagcccagagcggatctgtgccacagttcaagaaggtggtgttccaggagttcaccgatggaagcttcacccagccactgtaccggggagagctgaacgagcacctgggactgctgggaccatacatccgggccgaggtggaggataacatcatggtgaccttccggaaccaggccagccggccatacagcttctacagcagcctgatcagctacgaggaggatcagcggcagggagccgagccacggaagaacttcgtgaagccaaacgagaccaagacctacttctggaaggtgcagcaccacatggccccaaccaaggatgagttcgattgcaaggcctgggcctacttcagcgatgtggatctggagaaggatgtgcacagcggactgatcggaccactgctggtgtgccacaccaacaccctgaacccagcccacggacggcaggtgaccgtgcaggagttcgccctgttcttcaccatcttcgatgagaccaagagctggtacttcaccgagaacatggagcggaactgccgggccccttgcaacatccagatggaggatccaaccttcaaggagaactaccggttccacgccatcaacggatacatcatggataccctgccaggactggtgatggcccaggatcagcggatccggtggtacctgctgagcatgggaagcaacgagaacatccacagcatccacttcagcggacacgtgttcaccgtgcggaagaaggaggagtacaagatggccctgtacaacctgtacccaggagtgttcgagaccgtggagatgctgccaagcaaggccggaatctggcgggtggagtgcctgatcggagagcacctgcacgccggaatgagcaccctgttcctggtgtacagcaacaagtgccagaccccactgggaatggccagcggacacatccgggatttccagatcaccgccagcggacagtacggacagtgggccccaaagctggcccggctgcactacagcggaagcatcaacgcctggagcaccaaggagccattcagctggatcaaagtggatctgctggccccaatgatcatccacggaatcaagacccagggagcccggcagaagttcagcagcctgtacatcagccagttcatcatcatgtacagcctggatggaaagaagtggcagacctaccggggaaacagcaccggaaccctgatggtgttcttcggaaacgtggatagcagcggaatcaagcacaacatcttcaacccaccaatcatcgcccgatacatccggctgcacccaacccactacagcatcagaagcaccctgcggatggagctgatgggatgtgatctgaacagctgctccatgccactgggaatggagagcaaggccatcagcgatgcccagatcaccgccagcagctacttcaccaacatgttcgccacctggagcccaagcaaggcccggctgcacctgcagggacggagcaacgcctggcggccacaggtgaataacccaaaggagtggctgcaggtggatttccagaagaccatgaaggtgaccggagtgaccacccagggagtgaagagcctgctgactagcatgtatgtgaaggagttcctgatcagcagcagccaggatggacaccagtggaccctgttcttccagaacggaaaggtgaaggtgttccagggaaaccaggatagcttcaccccagtggtgaacagcctggatccaccactgctgacccgatacctgcggatccacccacagagctgggtgcaccagatcgccctgagaatggaggtgctgggatgcgaggcccaggatctgtactgatgagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 14 - DNA, 8004 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtgaggttaatttttaaactgtttgctctggttaataatctcaggaggttaatttttaaactgtttgctctggttaataatctcagggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgcagatcgagctgagcacctgcttcttcctgtgcctgctgcggttctgcttctccgccacccggcggtactacctgggagccgtggagctgagctgggattacatgcagagcgatctgggagagctgccagtggatgcccggttcccaccacgggtgccaaagagcttcccattcaacaccagcgtggtgtacaagaagaccctgttcgtggagttcaccgatcacctgttcaacatcgccaagccacggccaccctggatgggactgctgggaccaaccatccaggccgaggtgtacgataccgtggtgatcaccctgaagaacatggcctctcatcctgtgtccctgcacgccgtgggagtgagctactggaaggccagcgagggagccgagtacgatgatcagaccagccagcgggagaaggaggatgataaggtgttcccaggaggaagccacacctacgtgtggcaggtgctgaaggagaacggaccaatggccagcgatccactgtgcctgacctacagctacctgagccacgtggatctggtgaaggatctgaacagcggactgatcggagccctgctggtgtgccgggagggaagcctggccaaggagaagacccagaccctgcacaagttcatcctgctgttcgccgtgttcgatgagggaaagagctggcacagcgagaccaagaacagcctgatgcaggatcgggatgccgccagcgcccgggcctggccaaagatgcacaccgtgaacggatacgtgaaccggagcctgccaggactgatcggatgccaccggaagagcgtgtactggcacgtgatcggaatgggaaccaccccagaggtgcactctatcttcctggagggacacacctttctggtgcggaaccaccggcaggccagcctggagatcagcccaatcaccttcctgaccgcccagaccctgctgatggatctgggacagttcctgctgttctgccatatcagcagccaccagcacgatggaatggaggcctacgtgaaggtggatagctgcccagaggagccacagctgcggatgaagaacaacgaggaggccgaggattacgatgatgatctgaccgatagcgagatggatgtggtgcggttcgatgatgataacagcccaagcttcatccagatccggagcgtggccaagaagcacccaaagacctgggtgcactacatcgccgccgaggaggaggattgggattacgccccactggtgctggcccctgatgatcggagctacaagagccagtacctgaacaacggaccacagcggatcggacggaagtacaaaaaagtgcggttcatggcctacaccgatgagaccttcaagacccgggaggccatccagcacgagagcggaatcctgggaccactgctgtacggagaggtgggagataccctgctgatcatcttcaagaaccaggccagccggccatacaacatctacccacacggaatcaccgatgtgcggccactgtacagccggcggctgccaaagggagtgaagcacctgaaggatttcccaatcctgccaggagagatcttcaagtacaagtggacagtgacagtggaggatggaccaaccaagtctgatccaagatgcctgaccagatactacagcagctttgtgaacatggagagagacctggcctctggactgattggaccactgctgatctgctacaaggagtctgtggatcagagaggaaaccagatcatgtctgataagagaaatgtgatcctgttctctgtgtttgatgagaacagaagctggtacctgacagagaacatccagagattcctgccaaacccagccggagtgcagctggaggatccagagttccaggccagcaacatcatgcacagcatcaacggatacgtgttcgatagcctgcagctgagcgtgtgcctgcacgaggtggcctattggtatatcctgagcatcggagcccagaccgatttcctgagcgtgttcttcagcggatacaccttcaagcacaagatggtgtacgaggataccctgaccctgttcccattctccggagagaccgtgttcatgagcatggagaacccaggactgtggatcctgggatgccacaactctgatttcagaaacagaggaatgactgccctgctgaaagtgtccagctgtgataagaacactggagattactatgaggatagctatgaggatatctctgcctacctgctgagcaagaacaatgccattgagccaagaagcttcagccagaacccaccagtgctgaagagacaccagagagagatcaccagaaccaccctgcagtctgatcaggaggagattgattatgatgataccatctctgtggagatgaagaaggaggattttgatatctatgatgaggatgagaaccagagcccaagaagcttccagaagaagaccagacactacttcatcgctgcagtggagagactgtgggattatggaatgagcagcagcccacacgtgctgagaaacagagcccagagcggatctgtgccacagttcaagaaggtggtgttccaggagttcaccgatggaagcttcacccagccactgtaccggggagagctgaacgagcacctgggactgctgggaccatacatccgggccgaggtggaggataacatcatggtgaccttccggaaccaggccagccggccatacagcttctacagcagcctgatcagctacgaggaggatcagcggcagggagccgagccacggaagaacttcgtgaagccaaacgagaccaagacctacttctggaaggtgcagcaccacatggccccaaccaaggatgagttcgattgcaaggcctgggcctacttcagcgatgtggatctggagaaggatgtgcacagcggactgatcggaccactgctggtgtgccacaccaacaccctgaacccagcccacggacggcaggtgaccgtgcaggagttcgccctgttcttcaccatcttcgatgagaccaagagctggtacttcaccgagaacatggagcggaactgccgggccccttgcaacatccagatggaggatccaaccttcaaggagaactaccggttccacgccatcaacggatacatcatggataccctgccaggactggtgatggcccaggatcagcggatccggtggtacctgctgagcatgggaagcaacgagaacatccacagcatccacttcagcggacacgtgttcaccgtgcggaagaaggaggagtacaagatggccctgtacaacctgtacccaggagtgttcgagaccgtggagatgctgccaagcaaggccggaatctggcgggtggagtgcctgatcggagagcacctgcacgccggaatgagcaccctgttcctggtgtacagcaacaagtgccagaccccactgggaatggccagcggacacatccgggatttccagatcaccgccagcggacagtacggacagtgggccccaaagctggcccggctgcactacagcggaagcatcaacgcctggagcaccaaggagccattcagctggatcaaagtggatctgctggccccaatgatcatccacggaatcaagacccagggagcccggcagaagttcagcagcctgtacatcagccagttcatcatcatgtacagcctggatggaaagaagtggcagacctaccggggaaacagcaccggaaccctgatggtgttcttcggaaacgtggatagcagcggaatcaagcacaacatcttcaacccaccaatcatcgcccgatacatccggctgcacccaacccactacagcatcagaagcaccctgcggatggagctgatgggatgtgatctgaacagctgctccatgccactgggaatggagagcaaggccatcagcgatgcccagatcaccgccagcagctacttcaccaacatgttcgccacctggagcccaagcaaggcccggctgcacctgcagggacggagcaacgcctggcggccacaggtgaataacccaaaggagtggctgcaggtggatttccagaagaccatgaaggtgaccggagtgaccacccagggagtgaagagcctgctgactagcatgtatgtgaaggagttcctgatcagcagcagccaggatggacaccagtggaccctgttcttccagaacggaaaggtgaaggtgttccagggaaaccaggatagcttcaccccagtggtgaacagcctggatccaccactgctgacccgatacctgcggatccacccacagagctgggtgcaccagatcgccctgagaatggaggtgctgggatgcgaggcccaggatctgtactgatgagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 15 - DNA, 7948 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtggaattctggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagcggccgccaccatgcagatcgagctgagcacctgcttcttcctgtgcctgctgcggttctgcttctccgccacccggcggtactacctgggagccgtggagctgagctgggattacatgcagagcgatctgggagagctgccagtggatgcccggttcccaccacgggtgccaaagagcttcccattcaacaccagcgtggtgtacaagaagaccctgttcgtggagttcaccgatcacctgttcaacatcgccaagccacggccaccctggatgggactgctgggaccaaccatccaggccgaggtgtacgataccgtggtgatcaccctgaagaacatggcctctcatcctgtgtccctgcacgccgtgggagtgagctactggaaggccagcgagggagccgagtacgatgatcagaccagccagcgggagaaggaggatgataaggtgttcccaggaggaagccacacctacgtgtggcaggtgctgaaggagaacggaccaatggccagcgatccactgtgcctgacctacagctacctgagccacgtggatctggtgaaggatctgaacagcggactgatcggagccctgctggtgtgccgggagggaagcctggccaaggagaagacccagaccctgcacaagttcatcctgctgttcgccgtgttcgatgagggaaagagctggcacagcgagaccaagaacagcctgatgcaggatcgggatgccgccagcgcccgggcctggccaaagatgcacaccgtgaacggatacgtgaaccggagcctgccaggactgatcggatgccaccggaagagcgtgtactggcacgtgatcggaatgggaaccaccccagaggtgcactctatcttcctggagggacacacctttctggtgcggaaccaccggcaggccagcctggagatcagcccaatcaccttcctgaccgcccagaccctgctgatggatctgggacagttcctgctgttctgccatatcagcagccaccagcacgatggaatggaggcctacgtgaaggtggatagctgcccagaggagccacagctgcggatgaagaacaacgaggaggccgaggattacgatgatgatctgaccgatagcgagatggatgtggtgcggttcgatgatgataacagcccaagcttcatccagatccggagcgtggccaagaagcacccaaagacctgggtgcactacatcgccgccgaggaggaggattgggattacgccccactggtgctggcccctgatgatcggagctacaagagccagtacctgaacaacggaccacagcggatcggacggaagtacaaaaaagtgcggttcatggcctacaccgatgagaccttcaagacccgggaggccatccagcacgagagcggaatcctgggaccactgctgtacggagaggtgggagataccctgctgatcatcttcaagaaccaggccagccggccatacaacatctacccacacggaatcaccgatgtgcggccactgtacagccggcggctgccaaagggagtgaagcacctgaaggatttcccaatcctgccaggagagatcttcaagtacaagtggacagtgacagtggaggatggaccaaccaagtctgatccaagatgcctgaccagatactacagcagctttgtgaacatggagagagacctggcctctggactgattggaccactgctgatctgctacaaggagtctgtggatcagagaggaaaccagatcatgtctgataagagaaatgtgatcctgttctctgtgtttgatgagaacagaagctggtacctgacagagaacatccagagattcctgccaaacccagccggagtgcagctggaggatccagagttccaggccagcaacatcatgcacagcatcaacggatacgtgttcgatagcctgcagctgagcgtgtgcctgcacgaggtggcctattggtatatcctgagcatcggagcccagaccgatttcctgagcgtgttcttcagcggatacaccttcaagcacaagatggtgtacgaggataccctgaccctgttcccattctccggagagaccgtgttcatgagcatggagaacccaggactgtggatcctgggatgccacaactctgatttcagaaacagaggaatgactgccctgctgaaagtgtccagctgtgataagaacactggagattactatgaggatagctatgaggatatctctgcctacctgctgagcaagaacaatgccattgagccaagaagcttcagccagaacccaccagtgctgaagagacaccagagagagatcaccagaaccaccctgcagtctgatcaggaggagattgattatgatgataccatctctgtggagatgaagaaggaggattttgatatctatgatgaggatgagaaccagagcccaagaagcttccagaagaagaccagacactacttcatcgctgcagtggagagactgtgggattatggaatgagcagcagcccacacgtgctgagaaacagagcccagagcggatctgtgccacagttcaagaaggtggtgttccaggagttcaccgatggaagcttcacccagccactgtaccggggagagctgaacgagcacctgggactgctgggaccatacatccgggccgaggtggaggataacatcatggtgaccttccggaaccaggccagccggccatacagcttctacagcagcctgatcagctacgaggaggatcagcggcagggagccgagccacggaagaacttcgtgaagccaaacgagaccaagacctacttctggaaggtgcagcaccacatggccccaaccaaggatgagttcgattgcaaggcctgggcctacttcagcgatgtggatctggagaaggatgtgcacagcggactgatcggaccactgctggtgtgccacaccaacaccctgaacccagcccacggacggcaggtgaccgtgcaggagttcgccctgttcttcaccatcttcgatgagaccaagagctggtacttcaccgagaacatggagcggaactgccgggccccttgcaacatccagatggaggatccaaccttcaaggagaactaccggttccacgccatcaacggatacatcatggataccctgccaggactggtgatggcccaggatcagcggatccggtggtacctgctgagcatgggaagcaacgagaacatccacagcatccacttcagcggacacgtgttcaccgtgcggaagaaggaggagtacaagatggccctgtacaacctgtacccaggagtgttcgagaccgtggagatgctgccaagcaaggccggaatctggcgggtggagtgcctgatcggagagcacctgcacgccggaatgagcaccctgttcctggtgtacagcaacaagtgccagaccccactgggaatggccagcggacacatccgggatttccagatcaccgccagcggacagtacggacagtgggccccaaagctggcccggctgcactacagcggaagcatcaacgcctggagcaccaaggagccattcagctggatcaaagtggatctgctggccccaatgatcatccacggaatcaagacccagggagcccggcagaagttcagcagcctgtacatcagccagttcatcatcatgtacagcctggatggaaagaagtggcagacctaccggggaaacagcaccggaaccctgatggtgttcttcggaaacgtggatagcagcggaatcaagcacaacatcttcaacccaccaatcatcgcccgatacatccggctgcacccaacccactacagcatcagaagcaccctgcggatggagctgatgggatgtgatctgaacagctgctccatgccactgggaatggagagcaaggccatcagcgatgcccagatcaccgccagcagctacttcaccaacatgttcgccacctggagcccaagcaaggcccggctgcacctgcagggacggagcaacgcctggcggccacaggtgaataacccaaaggagtggctgcaggtggatttccagaagaccatgaaggtgaccggagtgaccacccagggagtgaagagcctgctgactagcatgtatgtgaaggagttcctgatcagcagcagccaggatggacaccagtggaccctgttcttccagaacggaaaggtgaaggtgttccagggaaaccaggatagcttcaccccagtggtgaacagcctggatccaccactgctgacccgatacctgcggatccacccacagagctgggtgcaccagatcgccctgagaatggaggtgctgggatgcgaggcccaggatctgtactgatgagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 16 - DNA, 8032 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtgaggttaatttttaaactgtttgctctggttaataatctcaggaggttaatttttaaactgtttgctctggttaataatctcagggaattctggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagcggccgccaccatgcagatcgagctgagcacctgcttcttcctgtgcctgctgcggttctgcttctccgccacccggcggtactacctgggagccgtggagctgagctgggattacatgcagagcgatctgggagagctgccagtggatgcccggttcccaccacgggtgccaaagagcttcccattcaacaccagcgtggtgtacaagaagaccctgttcgtggagttcaccgatcacctgttcaacatcgccaagccacggccaccctggatgggactgctgggaccaaccatccaggccgaggtgtacgataccgtggtgatcaccctgaagaacatggcctctcatcctgtgtccctgcacgccgtgggagtgagctactggaaggccagcgagggagccgagtacgatgatcagaccagccagcgggagaaggaggatgataaggtgttcccaggaggaagccacacctacgtgtggcaggtgctgaaggagaacggaccaatggccagcgatccactgtgcctgacctacagctacctgagccacgtggatctggtgaaggatctgaacagcggactgatcggagccctgctggtgtgccgggagggaagcctggccaaggagaagacccagaccctgcacaagttcatcctgctgttcgccgtgttcgatgagggaaagagctggcacagcgagaccaagaacagcctgatgcaggatcgggatgccgccagcgcccgggcctggccaaagatgcacaccgtgaacggatacgtgaaccggagcctgccaggactgatcggatgccaccggaagagcgtgtactggcacgtgatcggaatgggaaccaccccagaggtgcactctatcttcctggagggacacacctttctggtgcggaaccaccggcaggccagcctggagatcagcccaatcaccttcctgaccgcccagaccctgctgatggatctgggacagttcctgctgttctgccatatcagcagccaccagcacgatggaatggaggcctacgtgaaggtggatagctgcccagaggagccacagctgcggatgaagaacaacgaggaggccgaggattacgatgatgatctgaccgatagcgagatggatgtggtgcggttcgatgatgataacagcccaagcttcatccagatccggagcgtggccaagaagcacccaaagacctgggtgcactacatcgccgccgaggaggaggattgggattacgccccactggtgctggcccctgatgatcggagctacaagagccagtacctgaacaacggaccacagcggatcggacggaagtacaaaaaagtgcggttcatggcctacaccgatgagaccttcaagacccgggaggccatccagcacgagagcggaatcctgggaccactgctgtacggagaggtgggagataccctgctgatcatcttcaagaaccaggccagccggccatacaacatctacccacacggaatcaccgatgtgcggccactgtacagccggcggctgccaaagggagtgaagcacctgaaggatttcccaatcctgccaggagagatcttcaagtacaagtggacagtgacagtggaggatggaccaaccaagtctgatccaagatgcctgaccagatactacagcagctttgtgaacatggagagagacctggcctctggactgattggaccactgctgatctgctacaaggagtctgtggatcagagaggaaaccagatcatgtctgataagagaaatgtgatcctgttctctgtgtttgatgagaacagaagctggtacctgacagagaacatccagagattcctgccaaacccagccggagtgcagctggaggatccagagttccaggccagcaacatcatgcacagcatcaacggatacgtgttcgatagcctgcagctgagcgtgtgcctgcacgaggtggcctattggtatatcctgagcatcggagcccagaccgatttcctgagcgtgttcttcagcggatacaccttcaagcacaagatggtgtacgaggataccctgaccctgttcccattctccggagagaccgtgttcatgagcatggagaacccaggactgtggatcctgggatgccacaactctgatttcagaaacagaggaatgactgccctgctgaaagtgtccagctgtgataagaacactggagattactatgaggatagctatgaggatatctctgcctacctgctgagcaagaacaatgccattgagccaagaagcttcagccagaacccaccagtgctgaagagacaccagagagagatcaccagaaccaccctgcagtctgatcaggaggagattgattatgatgataccatctctgtggagatgaagaaggaggattttgatatctatgatgaggatgagaaccagagcccaagaagcttccagaagaagaccagacactacttcatcgctgcagtggagagactgtgggattatggaatgagcagcagcccacacgtgctgagaaacagagcccagagcggatctgtgccacagttcaagaaggtggtgttccaggagttcaccgatggaagcttcacccagccactgtaccggggagagctgaacgagcacctgggactgctgggaccatacatccgggccgaggtggaggataacatcatggtgaccttccggaaccaggccagccggccatacagcttctacagcagcctgatcagctacgaggaggatcagcggcagggagccgagccacggaagaacttcgtgaagccaaacgagaccaagacctacttctggaaggtgcagcaccacatggccccaaccaaggatgagttcgattgcaaggcctgggcctacttcagcgatgtggatctggagaaggatgtgcacagcggactgatcggaccactgctggtgtgccacaccaacaccctgaacccagcccacggacggcaggtgaccgtgcaggagttcgccctgttcttcaccatcttcgatgagaccaagagctggtacttcaccgagaacatggagcggaactgccgggccccttgcaacatccagatggaggatccaaccttcaaggagaactaccggttccacgccatcaacggatacatcatggataccctgccaggactggtgatggcccaggatcagcggatccggtggtacctgctgagcatgggaagcaacgagaacatccacagcatccacttcagcggacacgtgttcaccgtgcggaagaaggaggagtacaagatggccctgtacaacctgtacccaggagtgttcgagaccgtggagatgctgccaagcaaggccggaatctggcgggtggagtgcctgatcggagagcacctgcacgccggaatgagcaccctgttcctggtgtacagcaacaagtgccagaccccactgggaatggccagcggacacatccgggatttccagatcaccgccagcggacagtacggacagtgggccccaaagctggcccggctgcactacagcggaagcatcaacgcctggagcaccaaggagccattcagctggatcaaagtggatctgctggccccaatgatcatccacggaatcaagacccagggagcccggcagaagttcagcagcctgtacatcagccagttcatcatcatgtacagcctggatggaaagaagtggcagacctaccggggaaacagcaccggaaccctgatggtgttcttcggaaacgtggatagcagcggaatcaagcacaacatcttcaacccaccaatcatcgcccgatacatccggctgcacccaacccactacagcatcagaagcaccctgcggatggagctgatgggatgtgatctgaacagctgctccatgccactgggaatggagagcaaggccatcagcgatgcccagatcaccgccagcagctacttcaccaacatgttcgccacctggagcccaagcaaggcccggctgcacctgcagggacggagcaacgcctggcggccacaggtgaataacccaaaggagtggctgcaggtggatttccagaagaccatgaaggtgaccggagtgaccacccagggagtgaagagcctgctgactagcatgtatgtgaaggagttcctgatcagcagcagccaggatggacaccagtggaccctgttcttccagaacggaaaggtgaaggtgttccagggaaaccaggatagcttcaccccagtggtgaacagcctggatccaccactgctgacccgatacctgcggatccacccacagagctgggtgcaccagatcgccctgagaatggaggtgctgggatgcgaggcccaggatctgtactgatgagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 17 - protein, 738 aa, Unknown
  AAVhu.37 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 18 - protein, 738 aa, Unknown
  AAVrh.10 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 19 - DNA, 4371 bp, Artificial Sequence
  constructed sequence
atgcagatcgagctgtctacctgcttcttcctgtgcctgctgcggttctgcttcagcgccaccagacggtactatctgggcgccgtggaactgagctgggactacatgcagagcgacctgggcgagctgcccgtggacgccagattccctccaagagtgcccaagagcttccccttcaacacctccgtggtgtacaagaaaaccctgttcgtggaattcaccgaccacctgttcaatatcgccaagcccagacccccctggatgggcctgctgggacctacaattcaggccgaggtgtacgacaccgtcgtgatcaccctgaagaacatggccagccaccccgtgtctctgcacgccgtgggagtgtcctactggaaggcctctgagggcgccgagtacgacgatcagaccagccagcgcgagaaagaggacgacaaggtgttccctggcggcagccacacctacgtgtggcaggtgctgaaagaaaacggccccatggcctccgaccctctgtgcctgacatacagctacctgagccacgtggacctcgtgaaggacctgaacagcggcctgatcggagccctgctcgtgtgtagagagggcagcctggccaaagagaaaacccagaccctgcacaagttcatcctgctgttcgccgtgttcgacgagggcaagagctggcacagcgagacaaagaacagcctgatgcaggaccgggacgccgcctctgctagagcctggcccaaaatgcacaccgtgaacggctacgtgaacagaagcctgcccggactgatcggctgccaccggaagtctgtgtactggcacgtgatcggcatgggcaccacccctgaggtgcacagcatctttctggaaggacacacctttctcgtgcggaaccaccggcaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagaagctacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtactctagaaggctgcccaagggcgtgaaacacctgaaggacttccccatcctgcccggcgagatcttcaagtacaagtggaccgtgaccgtggaagatggccccaccaagagcgaccccagatgcctgacacggtactatagcagcttcgtgaacatggaacgggacctggcctccggcctgattggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccaaccctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcctgtggatcctgggctgccacaacagcgacttccggaacagaggcatgaccgccctgctgaaggtgtccagctgcgacaagaacaccggcgactactacgaggacagctatgaggacatcagcgcctacctgctgagcaagaacaacgccatcgagcccagaagcttcagccagaacccccccgtgctgaagcggcaccagagagagatcacccggaccaccctgcagtccgaccaggaagagatcgattacgacgacaccatcagcgtggaaatgaagaaagaagatttcgacatctacgacgaggacgagaaccagagcccccggtcctttcagaaaaagacccggcactacttcattgccgctgtggaacggctgtgggactacggcatgagcagcagccctcacgtgctgagaaacagggcccagagcggcagcgtgccccagttcaagaaagtggtgttccaggaattcacagacggcagcttcacccagcctctgtaccgcggcgagctgaacgagcacctgggactgctgggcccctatatcagagccgaagtggaagataacatcatggtcaccttccggaatcaggcctcccggccctacagcttctacagctccctgatcagctacgaagaggaccagagacagggcgctgagccccggaagaacttcgtgaagcccaacgagactaagacctacttttggaaggtgcagcaccacatggcccctacaaaggacgagttcgactgcaaggcctgggcctacttctccgacgtggacctggaaaaggacgtgcactctgggctgatcggccccctgctcgtgtgccacaccaacaccctgaatcccgcccacggcagacaggtgacagtgcaggaattcgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggagtgtctgatcggcgagcacctgcacgctgggatgagcacactgtttctggtgtacagcaacaagtgccagacacctctgggcatggcctctggccacatccgggactttcagatcacagccagcggccagtacggccagtgggccccaaaactggccagactgcactacagcggcagcatcaacgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactccagcggcattaagcacaacatcttcaacccccccatcattgcccggtacatccggctgcaccccacccactacagcatccggtccaccctgagaatggaactgatgggctgcgacctgaactcctgctccatgcccctggggatggaaagcaaggccatctccgacgcccagatcaccgcctccagctacttcaccaacatgttcgccacctggtccccatccaaggcccggctgcacctgcagggcagaagcaatgcttggaggcctcaggtgaacaaccccaaagagtggctgcaggtggacttccagaaaaccatgaaagtgaccggcgtgaccacccagggcgtgaagtctctgctgacctctatgtacgtgaaagagttcctgatctccagcagccaggacggccaccagtggaccctgtttttccagaacggcaaagtgaaagtgtttcaggggaaccaggactccttcacccccgtcgtgaatagcctggaccctccactgctgaccagatacctgcggatccaccctcagagttgggtgcaccagattgctctgcggatggaagtgctgggatgcgaggcccaggacctgtac

SEQ ID NO: 20 - protein, 736 aa, Unknown
  AAV3B capsid
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 21 - protein, 14 aa, Artificial Sequence
  Synthetic- 14 amino acid linker
SFSQNPPVLKRHQR

SEQ ID NO: 22 - protein, 738 aa, Adeno-associate virus hu.37; identical to SEQ ID NO: 17